(19) United States
(12) Patent Application Publication
(10) Pub. No.: US 2026/0020969 A1
(43) Pub. Date: Jan. 22, 2026

(54) WEARABLE ORTHOPEDIC TRACTION DEVICE FOR RELIEVING SHOULDER PAIN

(71) Applicants: Donald Abraham Volk, Honolulu, HI (US); Darryl Kan, Honolulu, HI (US); Neal EIAttrache, Honolulu, HI (US)

(72) Inventors: Donald Abraham Volk, Honolulu, HI (US); Darryl Kan, Honolulu, HI (US); Neal EIAttrache, Honolulu, HI (US)

(21) Appl. No.: 19/269,680

(22) Filed: Jul. 15, 2025

Related U.S. Application Data
(60) Provisional application No. 63/672,198, filed on Jul. 16, 2024.

(51) Int. Cl. A61F 5/01

(52) U.S. Cl. CPC A61F 5/013; A61F 2005/0132

(57) ABSTRACT

A brace assembly for applying traction to a shoulder of a user in a supine position that includes an arm brace configured to be secured to an arm homolateral to the shoulder of the user, a lower body anchor configured to be secured to a leg of the user, and an elastic cord having a first end secured to the arm brace at a near connection point and a second end secured to the lower body anchor at a far connection point. The elastic cord is configured to apply inferior traction to the upper arm, such that distractive force is transmitted to a shoulder joint of the shoulder. The arm brace is configured to position the near connection point adjacent to the elbow joint, thereby allowing forearm abduction and flexion of the elbow joint while preventing abduction of the upper arm and minimizing torque at the elbow joint during forearm abduction.

### CROSS-REFERENCE TO RELATED APPLICATIONS

This application claims the benefit of U.S. Provisional Application No. 63/672,198, filed on Jul. 16, 2024, the entirety of which is incorporated by reference herein.

### FIELD OF THE INVENTION

The present invention relates broadly to the field of therapeutic approaches and wearable devices for alleviating shoulder pain that may result from injury, surgical intervention, degenerative or inflammatory medical conditions, and/or chronic pain syndromes. It encompasses non-invasive orthopedic devices and methods designed to reduce discomfort and facilitate recovery in individuals suffering from a variety of shoulder pathologies. The invention is particularly suited for use during sleep or rest, where it replicates the biomechanical benefits of gravity on the shoulder when the body is upright, but in a manner adapted for the reclined or supine position

### BACKGROUND OF THE INVENTION

The shoulder joint, or glenohumeral joint, is a highly mobile ball-and-socket articulation that connects the humerus (upper arm bone) to the scapula (shoulder blade). It provides the widest range of motion of any joint in the human body, allowing for flexion, extension, abduction, adduction, circumduction, and internal and external rotation. This extensive mobility enables critical upper limb functions such as lifting and reaching. However, this mobility also comes at the cost of relative instability, which makes the shoulder susceptible to a wide array of injuries and degenerative conditions. When the shoulder joint is compromised-whether due to trauma, overuse, impingement syndrome, surgical recovery, or chronic and degenerative joint disease-normal biomechanics are disrupted.

This can lead to superior migration of the humeral head, increased friction and compression in the subacromial space, and eventual degeneration of joint surfaces and surrounding soft tissue structures. Such conditions frequently result in chronic pain, reduced range of motion, and functional impairment. Effective treatment often requires relieving the compressive forces within the shoulder joint and restoring proper anatomical spacing to prevent further damage and promote healing.

Shoulder pain resulting from these causes is particularly intensified during rest or sleep, especially in the supine or reclined position, resulting in compression of sensitive joint structures. Among the most affected populations are patients recovering from rotator cuff repair, labral reconstruction, and subacromial decompression procedures. In these cases, loss of downward gravitational forces acting upon the unsupported arm drive the humeral head superiorly, increasing contact pressure against healing tissues and producing pain that is often most severe at night. Similarly, patients suffering from adhesive capsulitis (frozen shoulder), thoracic outlet syndrome, or glenohumeral osteoarthritis frequently report night pain as their most limiting symptom, interfering with sleep and impeding recovery.

Conventional methods to manage shoulder pain during rest include the use of analgesic or anti-inflammatory medications, cryotherapy, physical therapy, and supportive devices such as pillows or positional bolsters. However, these approaches are often ineffective at maintaining the joint in a decompressed, neutral alignment that would reduce pain-causing mechanical stresses. In particular, they fail to simulate the joint-relieving effect of gravitational traction that occurs naturally when the body is upright and the arm hangs freely at the side.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the present invention there is provided a brace assembly for applying traction to a shoulder of a user in a supine position. The brace assembly includes an arm brace configured to be secured to an arm homolateral to the shoulder of the user, a lower body anchor configured to be secured to a leg of the user, and an elastic cord having a first end secured to the arm brace at a near connection point and a second end secured to the lower body anchor at a far connection point. The elastic cord is configured to apply inferior traction to the upper arm, whereby distractive force is transmitted to a shoulder joint of the shoulder. The arm brace is configured to position the near connection point adjacent to the elbow joint, thereby allowing forearm abduction and flexion of the elbow joint while preventing abduction of the upper arm and minimizing torque at the elbow joint during forearm abduction. The arm brace may include a main body portion that defines an arm aperture configured to receive a user's arm, where a connection extension extends outwardly from the main body portion, the main body portion includes a tightening system, and the near connection point is located on the connection extension. A support extension may extend outwardly from the main body portion of the arm brace in a direction opposite of the connection extension.

The connection extension may include first and second slots defined therein, and the elastic cord may extend through the first and second slots to connect the first end of the elastic cord to the connection extension. The main body portion of the arm brace may include an upper portion and a base portion, and the upper portion may be pivotable with respect to the base portion. The brace assembly may include a jacket that at least partially surrounds the main body portion, and that comprises a material that is under increasing tensile stress when a diameter of the arm aperture increases and transitions toward a neutral unstressed state when the diameter of the arm aperture decreases.

The main body portion of the arm brace may be a first circumferential member with a first arm aperture and the arm brace may include a second circumferential member that is spaced from the first circumferential member and that defines a second arm aperture. A bridge may extend between the first and second circumferential members, the first circumferential portion may include a first tightening system, and the second circumferential portion may include a second tightening system. The diameter of the main body portion may be adjustable to increase and decrease the diameter of the arm aperture and the arm brace may include an elastic inner liner positioned in the arm aperture and adjacent an inner surface of the main body portion. The inner liner may be under increasing compressive stress when a diameter of the arm aperture decreases and transition toward a neutral unstressed state when the diameter of the arm aperture increases. The inner liner may include first and second opposite ends, where a gap is defined between the first and second ends of the inner liner, and an insert is positioned in the gap.

The lower body anchor may be a foot stirrup that includes a strap member that defines a foot opening and includes a plantar portion. The elastic cord may be at least thirty inches in length in an unstretched state when the lower leg anchor is connected to the foot. The elastic cord may include at least a first cord tension identifying marker that is configured to be registered with a brace tension identifying marker. The elastic cord may have a traction tension value measured in pounds-force. A torque value measured in inch-pounds may be equal to or less than two times the traction tension value.

The brace assembly may include an arm brace configured to be secured to or positioned on the upper arm with the near connection point at or above the elbow joint, thereby allowing forearm abduction and flexion of the elbow joint while preventing abduction of the upper arm.

In accordance with a first aspect of the present invention there is provided a method of applying traction to a shoulder of a user in a supine position. The method includes securing an arm brace to an arm homolateral to the shoulder of the user, and securing a lower body anchor to a leg of the user. An elastic cord that includes first and second ends extends between the arm brace and the lower body anchor. The first end of the elastic cord connects to the arm brace at a near connection point that is located adjacent to an elbow joint of the user. The method also includes tensioning the elastic cord to apply inferior traction to an upper arm of the arm, whereby distractive force is transmitted to the shoulder joint. The elastic cord has a tension value. The tension value may be between about 3 lbf and about 9 lbf. The lower body anchor may be a foot stirrup, and the method may include inserting a foot of the leg through the foot opening and positioning the plantar portion below a bottom of the foot. The second end of the elastic cord may connect to the strap member at a point above a dorsal surface of the foot.

The arm brace includes a main body portion that extends around the arm. A connection extension may extend outwardly from the main body portion along the outer side of the arm and toward the elbow joint, and the near connection point is located on the connection extension. The connection extension may include first and second slots defined therein, and the elastic cord may include at least a first tension identifying marker thereon. The method may include extending the elastic cord through the first slot, registering the first tension identifying marker with the first slot, and extending the elastic cord through the second slot.

The method may be performed or relate to when the user has a shoulder condition or has had a shoulder surgery procedure. The shoulder condition or shoulder surgery procedure may be one of a rotator cuff allograft augmentation, a shoulder osteoarthritis procedure, a shoulder replacement, acromioclavicular joint reconstruction, impingement syndrome, adhesive capsulitis or thoracic outlet syndrome. The shoulder surgery procedure may be a rotator cuff allograft augmentation that includes a decellularized dermal allograft that has been positioned between a humeral head and an acromion of the shoulder. The application of inferior traction to the upper arm provides rostral distraction of the humeral head, thereby providing decompression of the decellularized dermal allograft from the acromion.

The shoulder condition may be thoracic outlet syndrome including compression of a brachial plexus in a thoracic outlet. The application of inferior traction to the upper arm may provide distraction of the thoracic outlet and decompression of the brachial plexus. The method may include abducting the forearm and flexing the elbow joint, whereby torque at the elbow joint is minimized and the arm brace prevents abduction of the upper arm. The method may include reducing tension on the elastic cord by flexing the leg.

The method may include registering the first cord tension identifying marker with a brace tension identifying marker when connecting the first end of the elastic cord at the near connection point prior to putting the arm brace on.

In accordance with an aspect of the invention there is provided an orthopedic brace for an arm or leg of a user that provides support for joints or muscles that includes a semi-rigid structure, padding, and a tightening system. The brace includes a main body portion that defines an aperture configured to receive the user's arm or leg. The brace is used to treat conditions selected from the group of fractures, ligament injuries, deformities, muscle or joint instability, contractures, or to reduce pain. The padding may include a) a compliant jacket that at least partially surrounds the main body portion and which is under increasing tensile stress when a diameter of the aperture increases and transitions toward a neutral unstressed state when the diameter of the aperture decreases, or b) a compliant liner adjacent an inner surface of the main body portion that is under increasing compressive stress when the diameter of the aperture decreases and transitions toward a neutral unstressed state when the diameter of the aperture increases, or c) both the compliant jacket of a) and the compliant liner of b). The tightening system is selected from the group of hook-and-loop, rack and pinion, ratchet buckle, cam lock, lace-and-pulley (e.g., Boa®), over-center lever or toggle clamp, ratchet strap with lever release, cord lock and cinch system, dial tightening system and tensioning band with slide adjuster.

One of the aims of the present invention is not to prevent abduction of the entire arm, a movement often associated with pain in the injured shoulder, but to allow and help enable natural, pain-free abduction or free movement of the forearm away from the midline of the body while still providing traction to the upper arm. It is this traction of the upper arm of the present invention, rather than restriction of movement of the forearm and hand, that results in effective inferior distraction of the shoulder joint. The device is designed to permit a full range of forearm motion and functional hand use, while maintaining therapeutic traction to the upper arm. Traction of the upper arm generally being the application of a pulling force to the upper arm to help align bones or relieve pressure (e.g., in the shoulder, as discussed herein).

The invention permits the far-point connection of the traction cord to be placed anywhere along the leg, with preferred placements below the knee and at the foot (which are each considered a part of the leg). Generally, securing the far-point connection of the traction cord anywhere on the leg (including the foot) may be referred to herein as a leg anchor or lower body anchor. In a preferred embodiment, a slip-on stirrup is fitted around or on the foot, with the posterior or bottom portion resting against the plantar surface and the cord attachment located above the dorsal (top) surface of the foot. This configuration takes advantage of the foot's natural tolerance for axial load and avoids the need for tight or invasive bindings around the foot or leg. Additionally, the longer length of the elastic cord enabled by a foot-based anchor leads to greater constancy of tension. Longer cords exhibit smaller changes in tension with equivalent changes in body position, making the traction force more stable and predictable. This extended path also allows for fine modulation of tension simply by bending or straightening the knee. The user can reduce traction by flexing the leg or increase it by extending the leg, enabling real-time adjustment of therapeutic force without removing the device.

The arm brace portion of the device is engineered to maintain stability under tension while providing comfort and anatomical conformity. Multiple brace embodiments allow for fine adjustment using hook-and-loop closures, ratcheting buckles, and/or a rack and pinion tightening system. Padding systems comprising either an expandable padded jacket, a compressible foam rubber inner liner, or both in combination, adjust dynamically with brace size and apply even circumferential pressure to prevent pressure points and slippage of the brace axially down the arm. In several embodiments, stabilizing extensions on the ulnar side of the forearm extending in the direction of the wrist prevent rotation of the brace and distribute load uniformly.

Taken together, these innovations provide a device that preserves anatomical function, minimizes stress and torque, enhances comfort, and delivers stable and therapeutically effective traction. Unlike prior art devices that immobilize the arm, stress vulnerable joints, and disrupt sleep, the present invention supports natural movement, respects the body's biomechanics, and provides relief precisely where it is needed, at the shoulder. It may be effective in treating the pain and postural complications associated with rotator cuff injuries and surgeries, osteoarthritis, adhesive capsulitis, thoracic outlet syndrome, and other shoulder conditions requiring prolonged shoulder unloading.

The present invention provides a therapeutic apparatus or brace assembly designed to alleviate shoulder pain through the application of gentle, sustained traction to the upper arm. This traction produces inferior distraction of the shoulder joint complex, decreasing mechanical compression between anatomical structures within the shoulder, particularly at the glenohumeral joint and subacromial space. The invention is intended to be used while the user is in a reclined or supine position, simulating the gravitational decompression typically experienced while upright. This positioning and force vector mimic the therapeutic effects of standing posture, but in a manner adapted for rest or sleep.

Inventors have discovered a broad and unexpected range of clinical indications that are addressed by the present invention, including postoperative pain from rotator cuff repair, labral repair, shoulder decompression procedures, atraumatic or degenerative shoulder impingement syndrome and adhesive capsulitis (frozen shoulder), thoracic outlet syndrome, glenohumeral osteoarthritis and rotator cuff allograft. These conditions often result in pain that is intensified at night due to compression of joint surfaces and soft tissues when lying flat, particularly when the arm is unsupported or positioned in ways that increase subacromial pressure. Traditional management with medication often provides only minimal relief. The shoulder traction device disclosed herein offers a physiologic, non-invasive, and mechanically adaptive solution for relieving such pain.

One or more embodiments of the present invention comprises a secondary aid for use in conjunction with the traction device described that further restores neutral positioning of the shoulder joint in a second plane of gravitational force. To compensate for the shift in gravitation force posteriorly that occurs when a person moves from an upright position to a supine position, which causes the shoulder to drop backward toward the support surface, a support cushion of specific dimensions is provided for placement beneath the shoulder and upper arm during recumbent use to restore anterior-posterior neutrality and resist retraction of the shoulder. This is especially important for individuals suffering with thoracic outlet syndrome, where symptoms are known to worsen when the shoulder is depressed posteriorly. While in the standing or seated position, the shoulder typically rests in a neutral position in the anterior-posterior axis. However, in the supine position, this posterior translation increases joint strain, alters the resting alignment of the glenohumeral joint, and exacerbates subacromial impingement and capsular tension.

The height and indention load deflection (ILD) of the support cushion are critical to achieving this neutral positioning and relieving pain. For individuals with an average body build, optimal support height is calculated as 3 to 4 percent of their body height in inches. For instance, a person standing 5 feet 10 inches (70 inches) would require a support cushion approximately 2.1 to 2.8 inches in height. The cushion's ILD, a standard measure of foam firmness, should preferably be in the range of 25 to 30 to provide adequate support without discomfort. When properly placed and dimensioned, the pillow offsets the posterior pull of gravity and supports the arm in a neutral scapular and glenohumeral orientation. This, in conjunction with the inferior distraction of the shoulder applied by the elastic cord, allows the shoulder to remain comfortably decompressed in all planes while the user sleeps or rests.

The arm brace portion of the invention serves as the proximal, or near-point (near with respect to the upper arm and shoulder), connection for the traction mechanism. It is designed to be worn around the upper forearm, near the elbow, or above the elbow on the upper arm, and functions as the anchor point for the elastic cord. The design intentionally avoids the wrist as a point of tractional force application, which has been shown to cause discomfort, tingling, and circulatory or nerve compression due to the concentration of neurovascular structures near the surface of the skin at the wrist. Wrist-based traction systems immobilize the arm in a fixed position along the body, which can create a psychological sensation of confinement or anxiety and prevent natural movement during sleep.

By anchoring the traction force at or near the elbow, the present invention allows the forearm and hand to remain free and mobile. This freedom permits the user to adopt different sleeping positions, including keeping the arm at the side or bringing it across the body with the elbow flexed. Such postural variability not only improves comfort but also helps prevent stiffness and preserves functional use of the limb, which is especially important during postoperative recovery. Furthermore, the elbow-proximate attachment minimizes the generation of mechanical torque during arm movement. For example, in a wrist-based system, as the user bends the elbow from a straightened position towards 90 degrees, the elastic cord length may increase significantly in length. For example, a cord length of 28 inches from the distal connection point on the lower body to the wrist elongates to greater than 38 inches. This elongation increases a cord tension set at 5.5 pounds-force to 9.3 pounds-force. Given a wrist-to-elbow distance of about 10 inches, generated torque will be up to 93 inch-pounds applied to the elbow joint, causing fatigue and strain. The present design eliminates this issue and reduces torque to a manageable or negligible level by positioning the point of cord tension at or near the elbow, which lies on or close to the axis of rotation. Using an example with the cord tension at 5.5 pounds, in a preferred embodiment, the limited or negligible torque at or on the user's elbow is equal to or less than double the tension (2*5.5 lbf equals 11 in-lbs). It will be appreciated that lbf may simply be referred to as pounds herein. Therefore, in a preferred embodiment, the elastic cord has a traction tension value measured in pounds-force (e.g., 5.5 lbf), and the torque value (measured in inch-pounds) is equal to or less than two times the traction tension value (e.g., 11 in-lbs).

The brace itself may take the form of a semi-rigid polymeric superstructure or a dense textile-based wrap. It is typically about two inches in width for a single adjustment mechanism design and 4 or more inches wide in total for a double adjustment mechanism design, and circumferentially adjustable from approximately 2.5 inches to 6 inches in diameter to fit a wide range of arm sizes. Adjustment mechanisms vary between embodiments and include a basic hook-and-loop strap system, a ratcheting buckle mechanism, and a rack and pinion system. However, these are not limiting and other types of adjustment systems are within the scope of the present invention. The rack and pinion system comprises a pair of circumferential straps actuated by a knurled or scalloped adjustment knob, allowing the user to fine-tune fit and compression with ease, even while wearing the device. The ratcheting buckle design offers a balance of simplicity and effective tightening, while the hook-and-loop system offers a lightweight and low-cost option.

A preferred embodiment of the brace includes a structural extension projecting a distance in the distal direction along the ulnar side of the forearm (e.g., approximately 3 inches). This extension serves several purposes: it distributes the pressure of the brace more evenly along the arm, enhances rotational stability, and resists twisting forces induced by the traction cord anchored near the upper end of the brace.

In another embodiment, this extension may continue distally all the way to the wrist and terminate in a wrist strap or pair of integrally formed “wings” that wrap circumferentially around the wrist and are secured using hook-and-loop fasteners or other fastening mechanism. These wings stabilize the distal brace, prevent rotation of the proximal brace portion induced by tension of the elastic cord, and further improve pressure distribution and brace conformity to the user's anatomy.

To enhance comfort, the brace may incorporate one of two integrated padding systems (or both). In one design, at least a portion of and possibly the entire brace is jacketed with a stretchable fabric-covered rubber layer such as neoprene between 2 mm and 8 mm thick, excluding the area occupied by the adjustment knob. When the brace is worn on a smaller arm and the circumference is minimized, the neoprene jacket is in a relaxed, unstretched state. As the brace expands to accommodate a larger arm, the neoprene jacket stretches accordingly, preserving a snug fit without folds or pressure points. In a second embodiment, the inner wall of the brace is lined with a compressible elastic liner made from a material such as nitrile butadiene rubber (NBR), ranging from 5 mm to 15 mm in thickness. This liner compresses circumferentially when the brace is tightened. Due to its elastic properties, the liner not only decreases in circumference but also expands inwardly in cross-section, effectively reducing the aperture diameter of the brace and conforming to the user's arm with greater surface contact. This ensures a secure, cushioned fit that adapts dynamically as the brace is tightened or loosened. A removable section of the inner liner, typically 10 mm to 40 mm in width, may be utilized to allow further adjustability. When inserted, the removable section accommodates a range of larger arm sizes; when removed, the brace can tighten to a much smaller diameter, making it suitable for pediatric or slender adult use.

In yet another embodiment, the brace may be divided into two linked sections, upper and lower or first and second, connected by rotatable pivot points located on either lateral side of the brace body, preferably below its midpoint. These pivot joints allow the upper, larger portion of the brace to swivel independently, conforming to the natural taper of the user's forearm and enhancing overall fit.

A further embodiment provides the brace with a conical shape and taper angle ranging from 1 to 15 degrees, offering improved pressure distribution and anatomical conformity over straight, cylindrical designs.

A further refinement applicable to all embodiments involves a separate structural extension, distinct from the ulnar projection, at the upper edge of the brace, oriented proximally toward the elbow. This extension (which is likely shorter than the ulnar extension or projection) projects approximately one to three inches and serves to bring the traction connection point as close as possible to the elbow joint's center of rotation. The elastic cord may be looped through two slots in this extension spaced approximately ⅜ inch apart, for example. The friction and tension of the elastic cord prevent slippage, maintaining the set cord length reliably during use.

In another embodiment, the brace is made wider to enhance structural support, distribute tightening forces more evenly, and reduce any localized pressure or twisting tendency. To accommodate this increased width, the brace may incorporate two tightening mechanisms (either rack and pinion or ratcheting buckle systems), spaced apart by a distance ranging from 1 inch to 4 inches. This configuration ensures even circumferential compression and ease of independent adjustment across the broader surface area.

The traction cord extends distally from the arm brace to a far-point connection on the user's lower body (far with respect to the upper arm or shoulder). This connection may be made at the thigh, calf, or anywhere along the leg, but is preferably located below the knee and most preferably at the foot. The far-point anchor may be a slip-on stirrup collar that wraps around and/or surrounds the foot and rests against the plantar surface. A position above or on the dorsal side of the foot serves as the connection site for the cord. This configuration leverages the natural load-bearing capacity of the plantar surface, allowing for secure, comfortable traction without the need for tight straps or adhesives. Moreover, anchoring the cord at the foot increases the overall length of the elastic cord, which has a beneficial effect on tension constancy. Longer elastic cords produce smaller changes in tension with movement, resulting in more consistent and stable force application. This consistency improves therapeutic effectiveness and user comfort. The connection point or points may be at the sides of the foot as well. The cord may also wrap around the foot. Preferably, the cord pulls from a point adjacent and/or above the dorsal side or top of the foot. Thus, “connection point” is essentially the point from where the traction cord pulls.

Furthermore, by anchoring to the foot, the user can modulate the amount of traction by simply flexing or extending the leg. Flexion of the knee decreases the cord length and reduces tension, while leg extension restores or increases it. This intuitive, movement-based control system enables the user to adjust traction in real time without disengaging the device.

In addition to its mechanical and ergonomic advantages, the present invention provides a clinically powerful therapeutic solution for managing specific and often difficult-to-treat shoulder conditions, as inventors have discovered, including, glenohumeral osteoarthritis, adhesive capsulitis (frozen shoulder), thoracic outlet syndrome, and pain resulting from rotator cuff allograft augmentation. The present invention includes methods or procedures for treating these or other conditions utilizing the brace assembly.

For example, in cases involving rotator cuff allograft augmentation with decellularized dermal grafts, the invention supports critical postoperative requirements for graft survival and incorporation. This procedure is typically indicated for large tears of the rotator cuff, measuring 3 cm or more in anterior-posterior width and/or medial retraction, or in patients with early-stage muscular atrophy (Goutallier stage 1 or 2), patients aged 70 or older, or those who participate in high-risk overhead sports or occupations involving repetitive shoulder strain. The graft is surgically positioned between the humeral head and the acromion, and its viability depends on the maintenance of a decompressed subacromial space. Without adequate spacing, the graft is vulnerable to compressive forces from the acromion, which can disrupt revascularization, induce shear stress, and impair healing.

The shoulder distraction device or brace assembly of the present invention may be used to help produce a controlled, low-level inferior force on the humerus, generating rostral separation between the humeral head and the acromion. This helps directly reestablish and maintain the acromiohumeral interval in a direction perpendicular to the acromial undersurface, thereby decompressing the graft and allowing for optimal biological integration during healing. The device mimics the natural gravitational separation present when upright, but enables it during rest and sleep, when positional compression would otherwise compromise graft success.

For glenohumeral osteoarthritis, in which joint pain arises from narrowing of the joint space and direct contact between the degraded articular surfaces of the humeral head and glenoid, the invention helps provide therapeutic decompression. The traction applied by the brace assembly acts in a vector generally parallel to the glenohumeral joint line, thereby opposing the vertical compressive forces that drive pain in osteoarthritic shoulders.

This inferior distraction reduces joint surface pressure, alleviates nociceptive input from bone-on-bone contact, and may reduce synovial inflammation by promoting mechanical offloading of the joint capsule and subchondral bone. Continuous or intermittent use of the device, particularly during periods of rest or sleep, may help prevent pain exacerbation due to posture-induced joint compression, and can support both conservative management and post-surgical recovery pathways.

Adhesive capsulitis, a condition marked by inflammation and subsequent fibrosis of the shoulder capsule, particularly the anterior-superior suspensory complex, may also be addressed by the present invention. As the joint capsule becomes thickened and inelastic, especially in the area of the coracohumeral and superior glenohumeral ligaments, the humeral head is pathologically drawn upward, resulting in loss of inferior glide and further aggravation of subacromial impingement. In its early stages, adhesive capsulitis is particularly painful at night, and this pain often goes poorly managed by medication alone.

The invention helps provide a gentle, tolerable level of continuous inferior traction on the homolateral arm that relieves this capsular suspension and allows the humeral head to settle downward, away from the acromion. By restoring or maintaining the subacromial and intra-articular spacing, the device helps reduce the pain associated with early capsular inflammation and helps preserve capsular elasticity. In later stages, where fibrosis limits motion, the same inferior distraction mechanism may assist in stretching the thickened capsule and mitigating secondary mechanical impingement. Because it can be used during sleep, the present invention directly addresses one of the most disabling aspects of adhesive capsulitis, rest-related pain and sleeplessness, without pharmacologic side effects.

Thoracic outlet syndrome, a neurovascular compression syndrome affecting the brachial plexus and subclavian vessels as they pass through the thoracic outlet, is also well suited for management with the present invention. TOS can be caused by various factors, including trauma, repetitive arm and shoulder movement, anatomical abnormalities, and even pregnancy. Compression occurs between the clavicle and first rib, in the scalene triangle, or beneath the pectoralis minor, and is often posture-dependent. Symptoms are worsened when the patient is in a supine position and in many cases when the shoulder is retracted posteriorly without posterior support under the shoulder and arm.

The shoulder traction device of the present invention provides a direct distraction of the thoracic outlet and decompression of the brachial plexus, thereby reducing neurogenic symptoms such as tingling, numbness, pain and weakness in the neck and affected limb. The device offers an effective therapeutic adjunct in both neurogenic and vascular variants of the condition. The support cushion previously mentioned provides further adjunctive relief by offsetting shoulder retraction and compression of the costoclavicular space by restoring anterior-posterior neutrality. Any of the shoulder related surgeries, issues, conditions, syndromes, etc. may collectively be referred to herein as shoulder conditions.

In all of the applications discussed herein, the device enables mechanical interventions tailored to the specific anatomical and physiological disruptions involved in each condition. Its non-invasive design, user-controlled adjustability, and sleep-compatible use make it an ideal candidate for incorporation into both conservative and postoperative treatment protocols.

Inventors have discovered a range of elastic cord tensions that provide shoulder pain relief based on body height and weight, as shown below. It is recommended to use the minimum tension required to provide pain relief. Notably, the tension requirement in pounds will decrease over time and during the healing process. Thus, in an embodiment of the invention, the cord may be replaceable or interchangeable so that different cords can be used over the course of treatment or different devices or brace assemblies, each with a differing cord tension, can be used over the course of treatment. The present invention includes methods or procedures for changing the cord tension over time.

TABLE 1

| | Height | Height | Height | Height |
|---|---|---|---|---|
| Body | 5′3″-5′6″ | 5′6″-5′ 9″ | 5′9″-6′0″ | 6′0′-6′3″ |
| Weight | Tension | Tension | Tension | Tension |
| 100-120 lbs. | 3.1-4.2 lbs. | 4.2-5.3 lbs. | 5.3-6.4 lbs. | 6.4-7.5 lbs. |
| 120-140 lbs. | 3.3-4.4 lbs. | 4.4-5.5 lbs. | 5.5-6.6 lbs. | 6.6-7.7 lbs. |
| 140-160 lbs. | 3.5-4.6 lbs. | 4.6-5.7 lbs. | 5.7-6.8 lbs. | 6.8-7.9 lbs. |
| 160-180 lbs. | 3.7-4.8 lbs. | 4.8-5.9 lbs. | 5.9-7.0 lbs. | 7.0-8.1 lbs. |
| 180-200 lbs. | 3.9-5.0 lbs. | 5.0-6.1 lbs. | 6.1-7.2 lbs. | 7.2-8.3 lbs. |
| 200-220 lbs. | 4.1-5.2 lbs. | 5.2-6.3 lbs. | 6.3-7.4 lbs. | 7.4-8.5 lbs. |
| 220-240 lbs. | 4.3-5.4 lbs. | 5.4-6.5 lbs. | 6.5-7.6 lbs. | 7.6-8.7 lbs. |
| 240-260 lbs. | 4.5-5.6 lbs. | 5.6-6.7 lbs. | 6.7-7.8 lbs. | 7.8-8.9 lbs. |

### BRIEF DESCRIPTION OF THE DRAWINGS

FIG. 1 is a top perspective view of a user in a supine position wearing an orthopedic traction device in accordance with the invention, with the user's arm bent and the elastic cord connected to a foot stirrup.

FIG. 2 is a top perspective view of a user in a supine position with the traction device applied, showing the arm in a straight position alongside the body and the elastic cord routed to a leg brace.

FIG. 3 is a schematic side view illustrating elbow flexion and comparative torque implications between positioning the arm brace at the wrist and near the elbow during arm raising.

FIG. 4 is a side view of the device worn by a user lying in a supine position with one leg bent, demonstrating a shortened cord path and the functional configuration of the system.

FIG. 5 is a perspective view of a preferred embodiment of the arm brace incorporating a rack and pinion tightening mechanism, an ulnar extension, and a proximal extension for elastic cord attachment.

FIG. 6 is a perspective view of an alternative embodiment of the arm brace featuring a ratchet buckle tightening system in place of the rotary knob.

FIG. 7 is a perspective view of an embodiment of the arm brace in which the elastic cord is secured directly to the brace via through-slots in a short extension.

FIG. 8 is a perspective view of a leg brace component of the device showing a textile wrap-around cuff with hook and loop fasteners and elastic cord attachment.

FIG. 9 is a perspective view of a foot stirrup anchor having a padded base and surrounding skirt for positioning the user's foot within the device.

FIG. 10 is a perspective view of an arm brace comprising two connected rack and pinion tightening mechanisms joined by a bridging structure to increase the contact area and improve stabilization.

FIG. 11 is a perspective view of an arm brace with a swiveling upper section that pivots relative to the lower section to allow angular conformance to forearm taper.

FIG. 12 is a perspective view of an arm brace similar to that of FIG. 11 but with a continuous lower section in place of the extension.

FIG. 13 is an exploded perspective view of the rack and pinion tightening assembly showing internal components including gear racks, rotary knob, spring, drive shaft, and housing interfaces.

FIG. 14 is a cross-sectional view of the joined upper and lower housing components of the rack and pinion system showing interlocking tabs and recesses.

FIG. 15 is a side perspective view of an alternative forearm brace embodiment having an upper compression section with a manually looped tightening strap and a wrist section with lateral wings.

FIG. 16 is a side elevational view of the forearm brace of FIG. 15 showing the extension structure, loop slot, hook and loop fasteners, and strap attachment details.

FIG. 17 is a perspective view of a further embodiment of the arm brace in the form of a textile cuff fitted on the upper arm and secured with both hook and loop and flip-over center latch mechanisms.

FIG. 18 is a detailed perspective view of the arm brace of FIG. 17 showing the flip-over center latch and hook and loop components.

FIG. 19 is a side perspective view of a dual rack and pinion arm brace with a continuous conical taper and a compliant foam liner positioned within the brace aperture.

FIG. 20 is a perspective view of a single rack and pinion brace fitted with a stretchable neoprene jacket, shown in a reduced diameter configuration.

FIG. 21 is a perspective view of the brace of FIG. 20 in an expanded configuration, showing the neoprene jacket in a stretched state around the enlarged circumference.

FIG. 22 is a cross-sectional front elevation view of a rack and pinion brace fitted with a compliant foam rubber liner and an optional insert, shown at maximum diameter.

FIG. 23 is a perspective view of the removable foam rubber insert used in the embodiment of FIG. 22, showing its separable layers.

FIG. 24 is a cross-sectional front elevation view of the brace of FIG. 22 shown without the removable insert and in a tightened configuration with the foam liner compressed.

FIG. 25 is a side view of a user in a supine position wearing a brace assembly and including a support cushion under the upper arm.

FIG. 26 is a perspective view of the support cushion of FIG. 25.

FIG. 27 is a perspective view of a brace in accordance with a preferred embodiment of the present invention.

FIG. 28 is a perspective view of the brace of FIG. 27 on a user's arm.

FIG. 29 is a perspective view of another brace in accordance with a preferred embodiment of the present invention.

FIG. 30 is a perspective view of an insert that can be used with the brace of FIG. 29.

FIG. 31 is a perspective view of a brace that includes an adjustable bridge in accordance with a preferred embodiment of the present invention.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

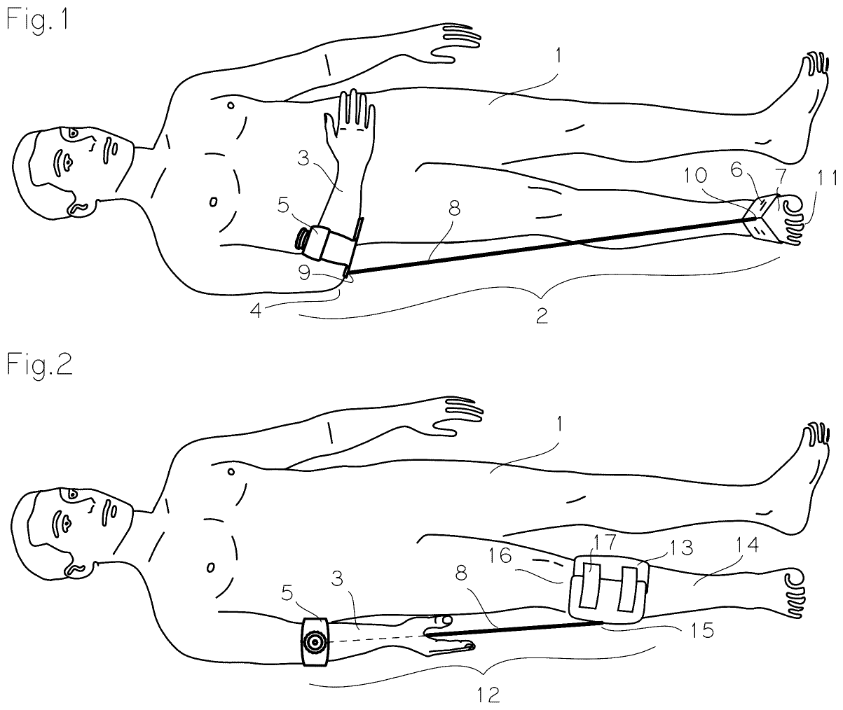

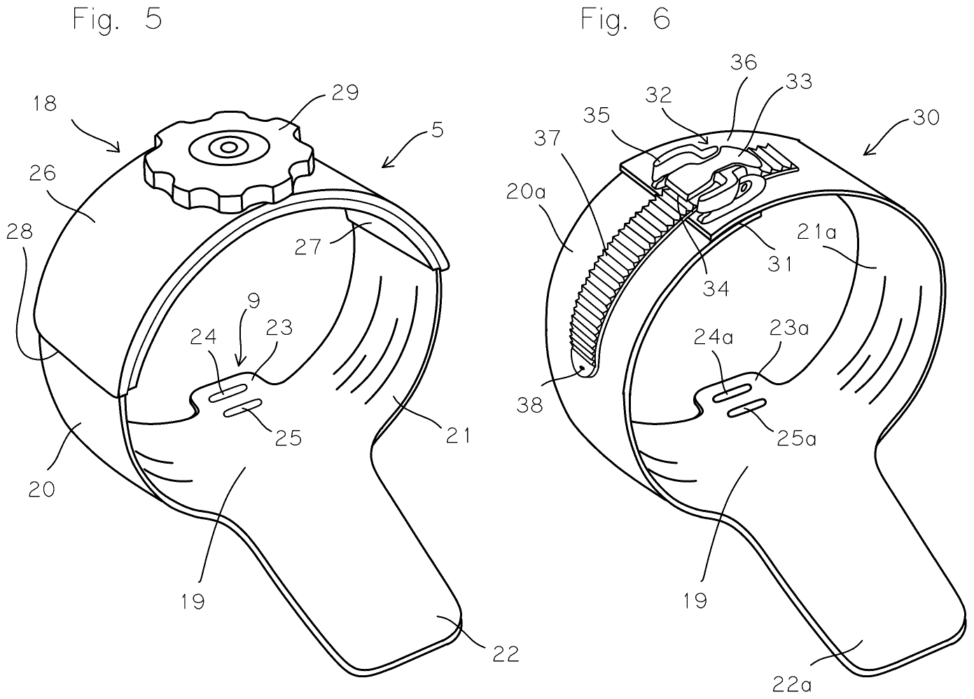

The present invention is directed to a therapeutic apparatus or brace assembly designed to alleviate shoulder pain through the application of gentle, sustained traction to the upper arm. FIGS. 1-31 illustrate various embodiments of the present invention. FIG. 1 illustrates a recumbent individual 1 lying in a supine position and wearing a first embodiment of the device or brace assembly 2. As shown in FIG. 1, the brace assembly 2 generally includes an arm member or arm brace 5, an elastic cord 8 and a lower body anchor or foot stirrup 6. In this configuration, the user's arm 3 is bent at the elbow 4 to an approximate angle of 80 degrees and crosses the torso at midsection level. The brace assembly 2 includes an arm brace 5 worn circumferentially around the upper forearm of the homolateral arm, i.e., the arm on the same side as the painful shoulder. A flexible elastic cord 8 is affixed proximally to arm brace 5 at connection location or point 9, which may be positioned adjacent to elbow 4 on the posterior or rear surface of arm 3. Notably, as shown in FIGS. 1 and 5, connection location 9 includes an extension portion or extension 23 protruding proximally from the main body portion of brace 5 in accordance with the invention, such that the elastic cord 8 is anchored as near as possible to the elbow's center of rotation. This strategic positioning minimizes torque during elbow flexion and ensures free, unrestricted movement of the forearm. However, this is not a limitation and the extension portion may be omitted.

The distal or opposite end of elastic cord 8 is attached to the lower body anchor, which may be a foot stirrup 6 at dorsal connection point 10, located just above the upper surface of the individual's foot 7. Foot stirrup 6 slips over the forefoot and around foot 7 and is designed such that its interior surface rests firmly against plantar surface 11 of foot 7. This placement allows the plantar arch—a naturally load-bearing surface—to absorb traction forces comfortably and without pressure-induced pain. The axial alignment of cord 8 between location 9 on brace 5 and location 10 on stirrup 6 provides inferior traction of the homolateral upper arm, producing therapeutic distraction of the glenohumeral joint while the user is at rest. It will be appreciated that any strap, stirrup, wrap or the like that allows a connection point for the elastic cord on the user's foot or at any other location on the leg or lower leg, in particular, is within the scope of the present invention.

Elastic cord 8 in this embodiment is preferably a flat elastic band approximately 11/16 inch wide and 3/16 inch thick or less, comprising an elastic rubber core enclosed within a protective woven sheath. This construction allows for strength, flexibility, and comfort, while resisting surface wear and minimizing user discomfort. Other forms of elastic cords—round, tubular, braided, or fabric-coated—may also be used within the scope of the invention. It will be appreciated that the dimensions and specific material are only exemplary and are not limiting. Any cord or band with elastic or stretchable properties is within the scope of the present invention.

FIG. 2 shows the same individual 1 in a reclined, supine position using a second embodiment of the device or brace assembly 12. In this configuration, the homolateral arm 3 is fully extended alongside the body in a straightened position. As in FIG. 1, arm brace 5 is secured to the upper forearm of arm 3. Elastic cord 8 is attached proximally to brace 5 at connection point 9 (not visible in this view), again located near elbow 4 and extending proximally from brace 5 to reduce torque and preserve elbow and forearm mobility.

In this embodiment, the distal end of elastic cord 8 is attached at connection location 15 on lower leg anchor or leg brace 13, which is fastened around the lower leg 14, just below knee joint 16. The upper edge of leg brace 13 preferably rests snugly against or adjacent to the inferior margin of knee 16 to provide a stable attachment point while allowing natural knee flexion and extension. Leg brace 13 is secured circumferentially around leg 14 using hook-and-loop fasteners 17, though it may alternatively include buckles, ratcheting clasps, or other adjustable tensioning systems.

As in FIG. 1, elastic cord 8 may comprise the preferred flat elastic band design, with the rubber core and protective sheath. Its length and flexibility enable it to maintain relatively constant tension during minor positional changes of the arm or leg, although to a lesser degree compared to the design shown in FIG. 1.

In combination, FIGS. 1 and 2 demonstrate a key functional advantage of the present invention: the ability to change arm posture freely, from a straight-arm configuration to a bent-elbow configuration (and any degree in between), without generating significant torque at the elbow joint. This is achieved through precise placement of the elastic cord's near-point connection 9 near the elbow's axis of rotation, enabling the user to move naturally and reposition the forearm or hand at will without altering the therapeutic effect or inducing fatigue. These features make the invention particularly suited for overnight use, where freedom of movement and the absence of mechanical resistance are essential for comfort and sustained therapeutic benefit.

It will be appreciated that both the foot-based anchor (FIG. 1) and the leg-based anchor (FIG. 2) create a generally axial traction vector from the upper arm toward the lower body, applying inferior force along the humerus and unloading the shoulder joint.

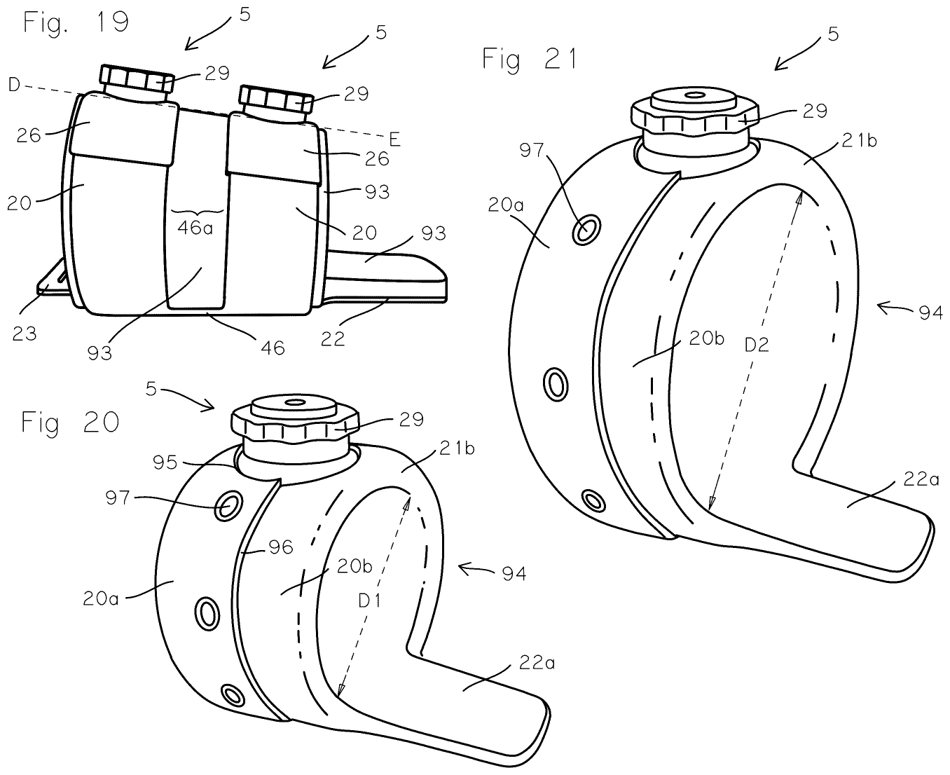

In all embodiments of the invention, the arm brace portions 5 of the brace assemblies of FIGS. 1 and 2, may incorporate a padding interface between the superstructure or main body portion of the arm brace and the arm to provide a comfortable and compliant fit. Two padded interface solutions are shown in detail in FIGS. 20-24, discussed further below.

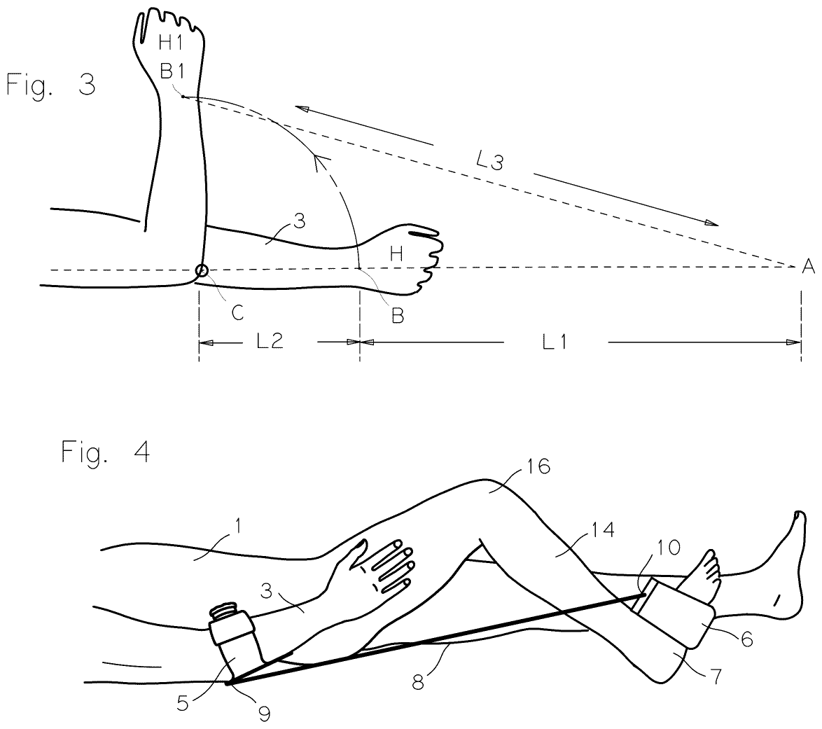

Referring now to FIG. 3, this diagram illustrates the biomechanical and geometric advantages of positioning the upper connection point of the cord adjacent the elbow joint. In FIG. 3, A represents the distal or lower (far-point) connection location of the traction cord 8, such as a point on the user's lower leg below the knee or at the foot. B represents the wrist location. C represents the olecranon, or elbow joint, which approximates the center of rotation for the forearm and the proximal attachment location (or near thereto) used in the present invention.

The horizontal arm with the hand in position H shows straight arm 3 positioned alongside the body and fully extended such that the direction of traction is colinear with the forearm axis, generating little to no torque, as the sine of the angle between the force vector and the lever arm is zero (sin 0°=0). The vertical arm with the hand in position H1 shows the elbow in a flexed position, with the forearm directed upward at 90 degrees, forming a right angle at the elbow joint C. Although the forearm is shown raised vertically in this depiction for illustrative clarity, in actual use, it is more common for the bent arm to lie flat across the torso, typically resting at a diagonal or cross-body angle over the user's midsection.

Dashed line A-B represents the cord length L1 between the wrist location B and distal connection point A. Dashed line A-C represents the cord length in the present invention, equal to L1+L2, where L2 is the length of the forearm between wrist B and elbow C. For illustrative purposes, L1 may be 28 inches and L2 may be 10 inches.

When the user's arm is in the straightened position (hand at H), and the cord is tensioned to a therapeutic level of 5.5 pounds, for example, both the prior art and present invention function similarly in terms of force application. However, when the elbow is flexed to 90 degrees and the hand moves to position H1, the consequences differ significantly between the two designs.

With the attachment point at wrist B, the movement to H1 and wrist location B1 increases the effective length of the cord path from L1 to L3, as represented by the diagonal line A-B1. Using the Pythagorean theorem, the increase in cord length is more than just the added forearm segment L2; it is approximately 11.29 inches longer than L1, leading to a significant increase in traction force from 5.5 pounds to approximately 9.3 pounds due to the elastic nature of the cord. This increased force is coupled with torque generation at the elbow C, calculated as 93 inch-pounds using the equation τ=F×r×sin(θ), where F=9.3 pounds, r=10 inches (distance from wrist to elbow), and θ=90°.

By contrast, in the present invention, the traction cord is attached proximally at location C, the elbow. As the forearm moves from the horizontal position (H) to the vertical position (H1), the distance A-C remains unchanged, and because the attachment point is approximately aligned with the pivot axis of the elbow, the effective length of the cord and its tension do not change. Consequently, there is no increase in traction force and little to no torque is generated (e.g., around or less than 10-12 inch-lbs). 10 inch-pounds or below is considered minimal or inconsequential torque. In use, with under 10 in-pounds, the wearer would notice little or no tension when flexing their elbow or abducting their forearm (meaning moving their forearm with respect to their upper arm, such as flexing the elbow). The inventors have determined that a torque value that is equal to or less than twice the tension of the cord is insignificant. Furthermore, since the forearm and hand are free to move without restriction, the user maintains full mobility and comfort. It will be appreciated that some torque may be generated and that zero torque may only be used herein for simplicity. In use, the user may connect the cord to a scale and then determine the tension. In an exemplary embodiment, the cord length from the elbow to the lower leg anchor when it is positioned below-the-knee (in a neutral or non-stretched state) may be equal to or greater than 20.1 inches. In an exemplary embodiment, the cord length from the elbow to the lower leg anchor when it is positioned on the foot (in a neutral or non-stretched state) may be equal to or greater than 29.6 inches. Forearm abduction may be defined as movement of the forearm away from alongside the body in the sagittal plane, when the forearm is raised anteriorly, and in a combination of sagittal and oblique or diagonal planes, when the forearm is drawn across the abdomen to rest over the anterior trunk, while the upper arm remains adducted and stabilized against the lateral thoracic wall.

FIG. 3 thus demonstrates how the present invention provides biomechanical function by eliminating unintended force amplification and mechanical strain during forearm movement. This allows for optimal and dynamic therapeutic use, particularly during rest or sleep, with two primary benefits: (1) freedom of forearm movement without altering therapeutic traction, and (2) elimination or reduction of torque generation at the elbow, which can cause fatigue and discomfort in wrist-based traction systems.

Referring now to FIG. 4, the drawing illustrates a user in a recumbent, supine position wearing the first embodiment of the present invention. In this configuration, individual 1 is shown with their leg 14 bent at the knee 16, demonstrating another feature of the system: user-controlled modulation of traction force through voluntary adjustment of leg position.

The user's homolateral arm 3 is depicted in a semi-bent position, secured by arm brace 5, which serves as the proximal anchor of the traction system. Connection point 9, located adjacent to the elbow, marks the attachment of elastic cord 8 to the brace. As with other embodiments of the invention, this proximal cord attachment near the elbow is positioned close to the joint's axis of rotation. As previously stated, this placement prevents and/or reduces torque generation during forearm motion and maintains a generally constant cord length, thereby allowing unrestricted forearm and hand movement without altering the tension applied to the upper arm. This feature enables the user to maintain functional mobility while receiving therapeutic traction.

At the distal or lower end, cord 8 attaches to foot stirrup 6 at connection point 10, located above the dorsal side of foot 7. The stirrup rests against the plantar surface of the foot and may comprise a slip-on design that provides secure yet comfortable anchoring. It will be appreciated that all of the drawings show the lower body anchor on the foot or leg on the same side of the body as the shoulder requiring traction. However, this is not a limitation and the lower body anchor can be positioned on the opposite leg or foot.

FIG. 4 highlights how bending the leg at the knee 16 reduces the cord path length, resulting in visibly reduced tension compared to when the leg is extended. This functionality allows the user to modulate traction force in real time, especially valuable when prolonged tension might lead to fatigue or discomfort. The tension can be temporarily decreased or even eliminated by bending the leg, and then easily reinstated by straightening it-offering dynamic pain management without needing to remove or readjust the device.

While FIG. 4 shows the knee raised above the resting surface, an alternative and often preferred posture involves turning the leg and knee outwardly so that both rest flat while remaining flexed, enabling full relaxation of the limb.

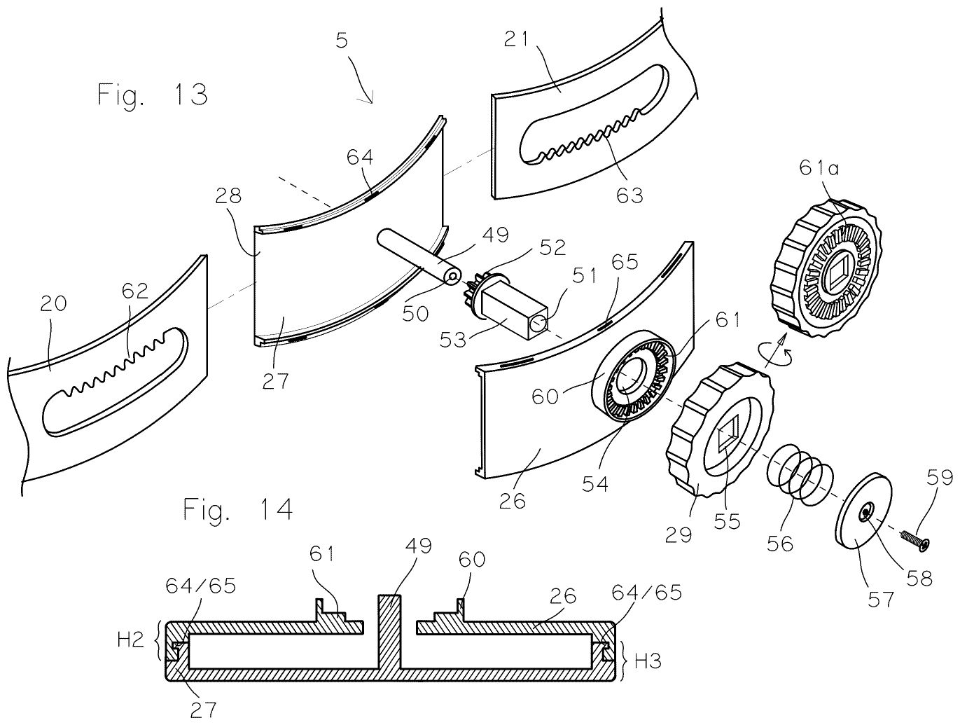

FIG. 5 shows arm brace 5 in more detail. This embodiment comprises a semi-rigid structural main body portion, circumferential member or superstructure 19 that encircles the user's arm and may be selectively tightened or loosened. In the embodiment shown in FIG. 5, the system for tightening or loosening is a rack-and-pinion tightening system 18 operated by a rotatable knob 29. The internal mechanics and gearing of this tightening system are illustrated in greater detail in FIGS. 13 and 14, but the external components and integration into the brace housing are clearly represented in the current figure.

The superstructure 19 is preferably formed from a durable injection moldable polymeric material that is dimensionally stable under applied loading forces. Suitable materials for the superstructure 19 include, but are not limited to, polypropylene (PP), acrylonitrile butadiene styrene (ABS), polyvinyl chloride (PVC), and polyoxymethylene (POM), among others. Of these, POM is preferred due to its superior mechanical strength and ability to withstand the substantial circumferential forces generated when the tightening knob 29 is rotated. Any suitable material is within the scope of the present invention.

The brace comprises a pair of strap sections 20 and 21, which extend circumferentially and meet at the base portion of the structure where they adjoin two integrated support extensions: a forearm support extension 22 and an elastic cord or connection point connection extension 23. These components together define the functional geometry of the brace's internal aperture and pressure distribution zones.

Support extension 22 projects outwardly from the main body portion (the portion that extends circumferentially around the user's arm) longitudinally along the ulnar side of the forearm toward the wrist. Its function is threefold: (1) to provide added surface area for distributing pressure generated by tightening of the brace; (2) to stabilize the brace and minimize axial slippage; and (3) to resist rotational or twisting forces that arise due to lateral tension from the elastic cord, which attaches at the opposite and shorter extension. In a preferred embodiment, extension 22 is approximately 2 inches wide and projects 3 inches from the circumferential strap structure. However, this is only exemplary.

Extension 23, which serves as the cord connection feature and point 9, projects outwardly from the opposite side of the main body portion in a direction toward the elbow. This positions the traction cord to apply its force proximate to the elbow joint to avoid torque generation and allow free movement of the forearm. Extension 23 is preferably 1 to 1.5 inches in length and may include one or more slots, such as pair of or first and second slots 24 and 25, through which the elastic cord is woven to form a secure, friction-locking loop. Other connection methods are within the scope of the present invention. Specifically, the cord enters through slot 24 from the outer surface of the brace and exits through slot 25, thus locking itself in place by tension-induced friction.

Slot 24 is positioned adjacent the elbow joint so that the traction force transmitted through the elastic cord is applied as close as possible to the center of rotation of the arm, in accordance with the objectives of the present invention. This placement ensures that cord tension does not vary substantially with forearm movement and produces little to no torque on the elbow.

The upper portion of the main body portion of the brace is occupied by the rack-and-pinion tightening mechanism 18, and includes an upper housing 26 and a lower housing 27 that together enclose the drive components. These two structures snap together using interlocking tabs on the lower housing 27 that engage with recesses molded into the interior of upper housing 26.

The upper housing 26 and lower housing 27 may each be manufactured from one of a variety of commonly used injection-molding materials, including PP, ABS, PVC, POM among many others. In this case, PP (polypropylene) is preferred for these components due to its high flexural compliance, which allows the assembled housing structure to conform more easily to the shape and size of the user's arm. A novel feature of the housing design is that upper and lower housings 26 and 27 are generally equal in overall height. In combination with the preferred use of compliant material, this geometry maximizes flexural adaptability of the entire tightening module, thus enabling the tightening mechanism to accommodate a wide range of arm sizes without encountering excessive resistance to bending.

The straps 20 and 21 extend through lateral openings 28 on either side of the tightening housing 26/27 and are operatively engaged with the internal rack components. Upon lifting and rotating knob 29, a pinion shaft is rotated, which drives the embedded rack teeth within straps 20 and 21 in opposite directions. Rotation of the knob in one direction draws the straps inward to tighten the brace, reducing the diameter of the central aperture and increasing circumferential pressure on the arm. Conversely, rotation in the opposite direction drives the straps outward to loosen the brace, enlarging the aperture and reducing pressure.

Once the desired fit is achieved, releasing the knob allows an internal spring mechanism to draw the knob downward, locking the pinion shaft in place. This spring-action locking feature ensures that the brace maintains its adjusted size and pressure without unintentional loosening during wear, thus preserving both comfort and therapeutic integrity.

Referring now to FIG. 6, another embodiment of an arm brace 30 is illustrated. This embodiment comprises a semi-rigid structural body or superstructure 19, which encircles the user's arm and is selectively tightened or loosened by a ratchet strap tightening system 32 that is operated via a ratcheting lever 33. Any type of tightening system may be used that allows the arm aperture to be made larger or smaller.

The superstructure 19 is preferably formed from a durable, injection moldable polymeric material that is dimensionally stable under tightening and traction loads. Suitable materials include polypropylene (PP), acrylonitrile butadiene styrene (ABS), polyvinyl chloride (PVC), and polyoxymethylene (POM), among others. In this embodiment, PVC is preferred for its optimal balance of stiffness, toughness, and manufacturability. Any suitable material is within the scope of the present invention.

Brace 30 includes a pair of circumferential strap sections 20a and 21a, which wrap around the user's arm and meet at the base of the brace, where they join two integrated extensions: a forearm support extension 22a and an elastic cord connection extension 23a. These extensions serve essential functional roles in defining the internal geometry of the brace and in evenly distributing compressive forces generated during tightening.

The forearm support extension 22a projects longitudinally along the ulnar side of the user's forearm in the direction of the wrist. As with the embodiment described in FIG. 5, this structural element provides increased surface area to distribute pressure from tightening forces applied by the ratchet mechanism. It also stabilizes the brace axially, reducing the risk of brace migration or slippage along the arm. Furthermore, it resists rotational or twisting forces that arise from lateral cord tension. As with previous embodiments, extension 22a measures approximately 2 inches in width and projects 3 inches distally from the central strap region for optimal support and stability.

The cord connection extension 23a is positioned on the medial side of the brace, in alignment with the elbow region. As in the prior embodiment, it includes slots 24a and 25a, which are functionally identical to slots 24 and 25 described in FIG. 5. The elastic cord is inserted through slot 24a from the outer surface, then looped internally and directed out through slot 25a, creating a secure, non-slip fixation by means of tension and friction.

The upper portion of the main body portion of the brace 30 houses the ratchet tightening mechanism 32, which comprises a lever arm 33, an internal pawl 34, opposed release arms 35 on either side, and a ratchet strap rack 37. The ratchet mechanism 32 is mounted on the exterior of the brace at location 36, and may be fixed in place using snap fits, rivets, ultrasonic welding, or other means. The ratchet strap 37 is affixed to strap section 20a at location 38 by similar mounting means. The strap 37 includes integral gear teeth for ratchet engagement and is preferably manufactured from a rubberized polymer that offers the necessary flexural adaptability to conform to the brace and withstand the tightening forces required to secure the arm.

In operation, the user lifts and pumps lever 33, which incrementally tightens the brace by drawing strap 21a across and over strap 20a at interface region 31. This motion progressively reduces the diameter of the internal aperture, resulting in a snug fit around the user's arm. The use of a mechanical advantage lever allows for fine-tuned adjustment with minimal applied effort, and the incremental locking effect of the pawl and ratchet combination ensures that the tension is preserved without slippage.

To loosen or remove the brace, the user simply presses one of the release arms 35, which disengages the pawl 34 from the ratchet rack 37. This allows the strap to slide freely and the brace to be removed or repositioned. Ratchet systems of this type are widely used in ski boots, inline skates and some wearable medical supports, offering proven reliability and robust locking characteristics. In the present invention, this mechanism allows the brace to be applied quickly and with precision, providing firm, adjustable retention around the arm while supporting the therapeutic traction function of the overall system.

As with prior embodiments of the brace described herein, the placement of cord extension 23a adjacent the elbow ensures that the traction cord applies force close to the axis of elbow rotation. This configuration eliminates and/or reduces torque generation during arm movement and allows freedom of forearm and hand motion.

Referring now to FIG. 7, a perspective view of brace 5 is shown, illustrating one way in which the elastic cord 8 may be attached to the cord connection extension 23 (at cord connection point or location) of the brace in the first embodiment of the present invention. As illustrated, elastic cord 8 is inserted through slot 24 from the outer surface of the brace extension 23. Once passed through slot 24, the cord routes over an internal bridge formed between the slots on extension 23 where it forms loop 39, and is then threaded outwardly through slot 25. This double-pass looped configuration, in which the cord passes through both slots and wraps around the internal bridge, creates a mechanically stable and frictionally secure attachment. In a preferred embodiment, the cord 8 includes one or more cord tension identifying markers 8a thereon. The cord tension identifying markers relate to the height of the user. For example, the cord length between the near and far point connections has to be longer for a taller user. Therefore, when assembling the brace assembly, the user can position the proper tension identifying marker in the proper position on the arm brace. For example, as shown in FIG. 7, the cord tension identifying marker 8a is lined up or registered with the first slot 24 (which may be referred to as a brace tension identifying marker). There may be any amount of cord tension identifying markers on the cord (e.g., one for each inch of height). In another embodiment, the arm brace may include a mark, indicia or brace tension identifying marker thereon with which to align or register the tension identifying marker on the cord.

The configuration ensures that any tensile loading of the cord, whether from the inherent tension generated during therapeutic use, or from incidental tugging, pulling, or arm movement, acts to tighten the loop and press the cord into the slots more firmly. Because the connection point lies adjacent to the elbow and near the axis of elbow rotation, the securement method shown in this figure preserves the biomechanical benefits of the invention, namely, torque-free forearm movement and consistent tractional force application. The tension identifying marker(s) can be on the lower body anchor as well.

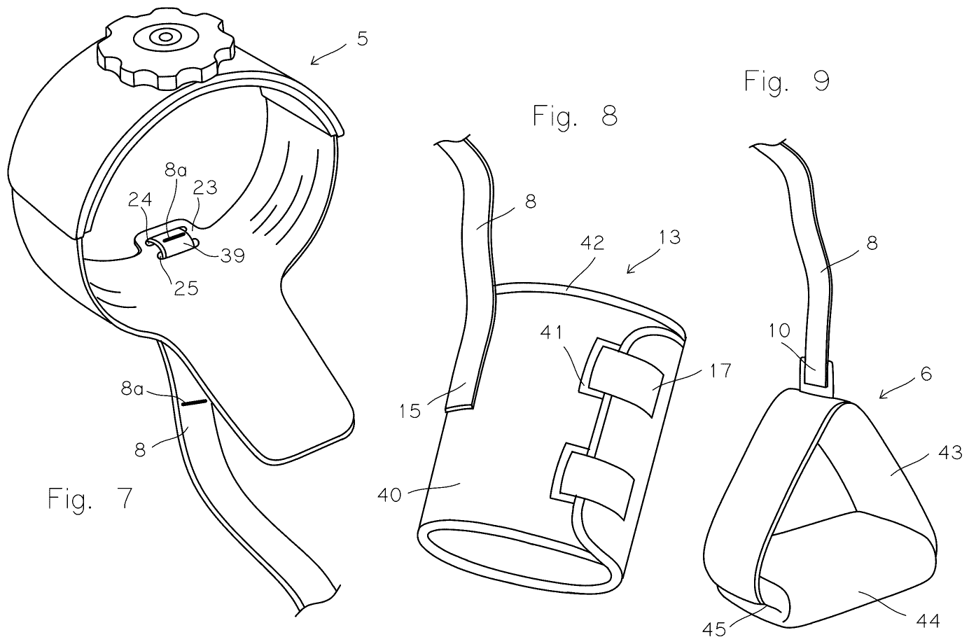

Referring to FIG. 8, a perspective view of a far-point connection brace, designated generally as brace 13, is depicted. Brace 13 is configured to be applied to the leg, preferably just below the knee (but at any point on the lower leg), and serves as the distal anchor point for the elastic cord that provides therapeutic shoulder traction in the present invention.

Brace 13 comprises a wrap-style body 40 or main body portion, made from a flexible textile laminate that includes a foam-padded interior layer to provide both structure and cushioned comfort. The laminated foam interface adds thickness and compressibility, helping to distribute the applied load over a broader surface area and preventing pressure points. Any type of wrap, band, strap or the like that allows connection to the leg is within the scope of the invention.

The brace is designed to wrap or extend circumferentially around the user's leg, particularly the upper calf area just distal to the patella. Once in position, the brace is secured using a pair of hook-and-loop fastening systems, which comprise hook straps 17 attached to the overlapping flap portion of wrap 40 and corresponding loop pads 41 affixed to the underlying layer. The hook and loop materials may be reversed. The presence of two fastening zones ensures better load distribution and resists rotation or loosening during leg movement. However, this is not limiting and only a single fastening zone may be used. Other connection or securing systems or methods are within the scope of the present invention (e.g., snaps, latches, clasps, buttons, etc.).

The proximal edge 42 or upper edge or surface of the brace, defined as the uppermost portion when worn, rests directly beneath the user's knee joint, helping to prevent upward migration of the brace during use. The elastic cord 8, which provides the traction component of the invention, is connected to brace 13 at attachment point 15. This connection may be made through sewn, riveted, or adhesive bonding techniques, or by using an integrally molded or stitched loop embedded into the wrap body. In the illustrated embodiment, the cord emerges from the side of the brace, indicating a preferred lateral routing path that avoids interference with the user's body. In practice, the user wraps brace material 40 around the leg below the knee, tightens the wrap to a comfortable level, and secures it in place using the two hook-and-loop fastening zones.

Referring now to FIG. 9, a detailed perspective view is shown of foot stirrup 6, previously shown in FIG. 1 and now further refined with additional structural detail. This component functions as the distal anchoring element of the traction system and is designed to interface with either the homolateral or opposite foot from which tractional force is applied during therapeutic use of the device.

Foot stirrup 6 comprises a skirt section or strap member 43, which wraps fully around the dorsal and lateral surfaces of the user's foot and includes a plantar portion. The foot can be inserted through a foot opening defined in the foot stirrup and/or strap member 43. The plantar portion of the strap member 43 may include a base pad 44, which is designed to rest flush against the plantar surface of the foot. The strap member 43 is preferably made of a durable and compliant material, such as heavy-gauge nylon webbing, thermoplastic elastomeric mesh, ballistic fabric, or neoprene-backed textiles, all of which offer strength, resilience, and moderate flexibility to accommodate varying foot geometries while contributing to a stirrup design that easily slips over the foot.

Plantar portion or pad 44 may be composed of a breathable material such as a cloth-covered outer layer over a foam core, where the core may include open-cell polyurethane foam, cross-linked polyethylene (XLPE), ethylene-vinyl acetate (EVA) foam, or TPE-based foam, among other formulations. These materials are selected for their ability to dissipate heat, resist compression, and offer consistent rebound properties under load. However, any suitable material is within the scope of the present invention. The thickness of pad 44 may range from approximately ⅜ inch to ⅝ inch, while its width may range from 4 to 5 inches and depth from 2 to 3 inches, providing enough contact area for both comfort and stability. These dimensions are only exemplary and not limiting. The foam material preferably does not trap heat and may be perforated to avoid heat build-up or perspiration during prolonged use. Slot 45 provides a passageway within pad 44 through which the plantar portion of the strap member 43 passes. In another embodiment, the plantar portion and the skirt may be a loop that is wrapped around the user's foot. To further enhance the mechanical stability and load distribution properties of the stirrup, a semi-rigid and generally flat insert may be placed beneath the top layer of foam core of pad 44 and within slot 45. This insert is preferably made from a thin polymeric sheet, such as thermoplastic polypropylene, HDPE or ABS, sized to match the footprint of the pad. The insert provides a semi-rigid and supportive under-layer that additionally provides a beneficial amount of flexural compliance for plantar support and user comfort. This semi-rigid element prevents unwanted deformation or buckling of the pad under load, thereby maintaining uniform pressure against the plantar surface and enhancing the effectiveness of the device.

Elastic cord 8 connects to foot stirrup 6 at attachment point 10, which may comprise a sewn loop, D-ring, molded clip, or stitched sleeve that ensures a secure interface between the cord and the stirrup. The positioning and orientation of elastic cord 8 ensure that tractional forces are transmitted axially along the body when the user is in a supine position, thus contributing to shoulder joint distraction in accordance with the therapeutic goals of the invention. Together, the structural features of foot stirrup 6 provide a reliable, cushioned, and anatomically compatible interface that securely transmits tractional force from the elastic cord to the user's painful shoulder while providing a distal anchor that is very easy to apply and remove.

Referring to FIG. 10, a further embodiment of the invention is shown in an arm brace 5c with two main body portions 5a and 5b or two circumferential members with knob-controlled rack-and-pinion tightening systems 29 are mechanically joined to function as a single integrated unit (designated as arm brace 5c). This configuration expands the effective contact and compression area of the device around the user's arm, thereby improving fit, pressure distribution, and anti-rotation stability.

In this embodiment, each rack-and-pinion unit includes strap sections 20 and 21 and a corresponding base portion 19a. The two base portions 19a are joined by an integral superstructure bridge 46, which connects the two units and causes them to operate in a coordinated yet independently adjustable manner. The union of the two sections forms a single semi-rigid device with dual independent tightening zones, each driven by its own knob system. This allows for differential adjustment along the upper and lower regions of the arm to better accommodate anatomical variations and user-specific needs.

One of the primary advantages of this dual-knob arrangement is the increased contact area between the brace and the arm, which allows compressive forces to be spread across a wider surface. This results in a more secure, comfortable, and evenly distributed fit while also minimizing localized pressure points that may occur with a single tightening mechanism. Furthermore, the larger surface area reduces the likelihood of rotational or axial slippage, which can be caused by tension forces from the connected elastic cord during dynamic use.

The upper portion of the integrated brace structure includes elastic cord connection extension 23, which is situated on the more proximal of the two knob-controlled brace sections. This portion is designed to interface with the upper portion of the forearm, which tends to have a larger circumference. Conversely, the lower portion of the dual-brace configuration includes ulnar support extension 22, which lies closer to the wrist and is designed for a narrower forearm profile, following the natural taper of the arm from elbow to wrist. Due to the extended length of the overall brace structure in this embodiment, the length of extension 22 may be shortened compared to the single-brace design, or even eliminated, as its supporting function is already partially shared by the adjoining upper unit.

The two brace assemblies are spaced apart by an intermediate gap 46a, which adds to the overall length of the device and allows each tightening system to work independently. This space contributes to the axial length needed for an effective and stable wrap and is particularly advantageous for users with longer forearms or larger arms in general. The gap 46a can vary in size depending on design or customization needs, with a typical range extending from approximately ¼ inch to over 2 inches. However, this dimension is only exemplary. The combined assembly is thus both structurally integrated and functionally modular, allowing enhanced adjustability, security, and user comfort. This dual-brace configuration allows for ergonomic conformity and customization, by adapting to the natural taper and curvature of the user's forearm, and allowing for localized tightening via the two independent knob systems.

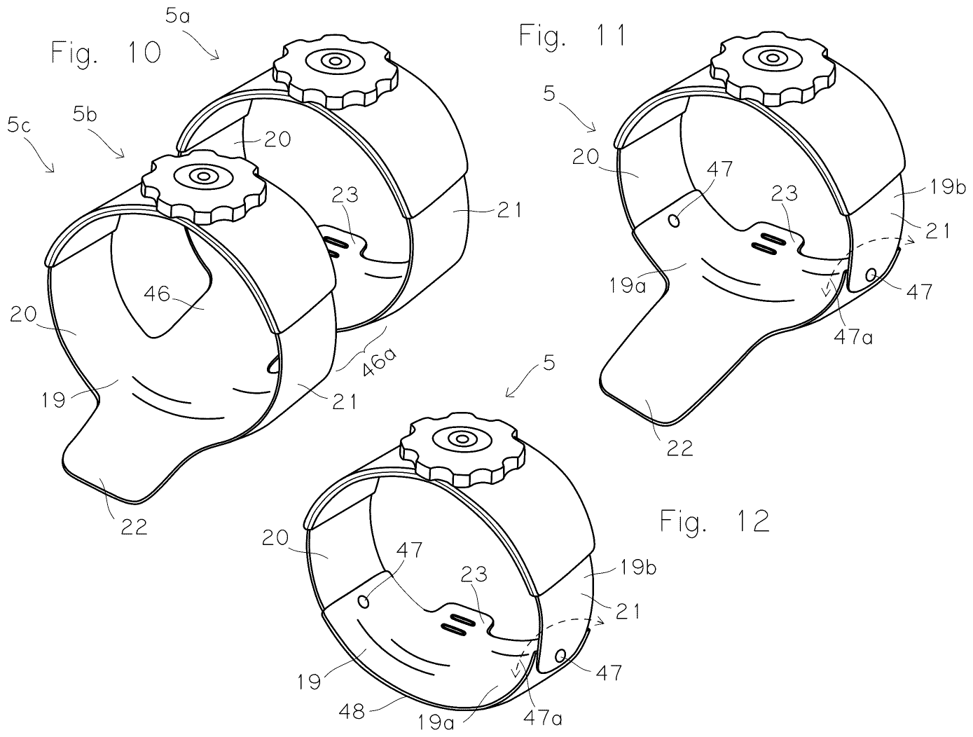

Referring to FIG. 11, another embodiment of the arm brace 5 of the present invention is shown, which includes a knob-controlled rack and pinion tightening system mounted to an upper brace section or upper portion 19b (that may include circumferential straps 20 and 21) and a base portion 19a of the main body portion. This configuration features a swiveling structural design wherein the upper tightening assembly and upper portion of the brace pivots independently from the lower base portion 19a of the brace.

The upper section comprising straps 20 and 21 is rotatably joined to the lower base 19a through pivot or rotation points 47, which are located on each side of the brace structure. These rotation points allow the upper assembly to swivel in an arcuate path, as indicated by dotted arc line 47a. This pivotal connection permits the brace to conform more effectively to the natural taper and shape of the human forearm, which generally is not cylindrical and typically exhibits a taper no greater than 7 to 10 degrees. The upper portion of the main body portion pivots with respect to the lower base portion.

The brace components are preferably formed of semi-rigid, structurally flexible materials, and because padding is incorporated in all embodiments of the invention, the placement of the rotation points 47 slightly below the midline of the formed aperture does not hinder the rotational functionality of the upper section. Instead, it facilitates dynamic adjustment and adaptive fit. A rivet or rotational shaft placed through aligned holes in the overlapping regions of strap ends 20 and 21 and base 19a provides the necessary structural integrity and rotational function for this pivotal joint.

This adaptable configuration enables the brace to accommodate variations in forearm contour and reduces localized compression that would otherwise result from attempting to force a cylindrical brace onto a conically tapered limb. This feature allows for even pressure distribution, which translates to less perceived tightness and enhanced comfort for the user. In addition, it helps to prevent axial slippage, which could otherwise occur during dynamic use of the traction device.

Of further functional significance, the location of the elastic cord connection extension 23 on a pivotable portion of the brace means that traction forces applied through the cord do not induce unwanted twisting of the entire brace. Instead, the upper section remains adaptive to the upper forearm, while the lower section maintains a stable position against the ulnar side of the arm, thereby preserving overall balance and comfort. This embodiment thus provides an advanced fit-optimization mechanism that enhances the effectiveness and usability of the brace by allowing articulated conformity to anatomical geometry while maintaining the securement, torque-resistance, and free movement benefits central to the invention.

Referring to FIG. 12, another embodiment of the arm brace is shown, which incorporates the knob-controlled rack and pinion tightening system, similar in overall design and functionality to the embodiment of FIG. 11, with the exception that the ulnar support extension 22 has been omitted. In this configuration, the brace comprises a semi-rigid lower base portion 19a and circumferential straps 20 and 21, which collectively form the body of the brace. The upper tightening assembly, composed of straps 20 and 21, is joined to the lower body 19a through a pair of pivoting rotation points 47, which permit the upper section to swivel independently of the base. The swivel path of the upper section is illustrated by dotted arc line 47a, allowing the device to conform to varying forearm tapers for improved fit and pressure distribution.

Notably, this embodiment does not include a projecting extension for support along the ulnar side of the forearm. Instead, the lower base portion 19a continues as a smooth, uninterrupted curve through region 48, where an extension, such as that seen in FIG. 11, would otherwise be located. This results in a more compact form factor, which may be advantageous in certain clinical or anatomical scenarios, such as when there is limited available space on the user's arm or when minimizing device footprint is desired.

While the absence of the ulnar extension reduces the brace's ability to distribute pressure over a broader forearm surface and diminishes resistance to twisting caused by lateral traction forces from the elastic cord (attached at connection extension 23), the embodiment shown in FIG. 12 retains all other core benefits and may be used when a minimal footprint or reduced coverage is preferred.

FIG. 13 presents an exploded perspective view of the internal components comprising the knob-controlled rack and pinion tightening system, which functions as the adjustment mechanism in the brace designs shown in some of the previous figures.

In the illustrated embodiment, strap 20 and strap 21 each include integrally formed gear racks 62 and 63, respectively. These racks are configured to engage with a central pinion gear 52, which is molded as a single unit with a square drive shaft 53. During assembly, straps 20 and 21 pass over post 49 for placement into the lower housing 27, seated against the inner surface of the housing and occupying space or opening(s) 28. Integrated pinion gear 52 and drive shaft 53 are inserted onto post 49 through hole 51. In this configuration, the teeth of the pinion gear mesh precisely with the opposing racks 62 and 63 such that rotation of the shaft results in the symmetric advancement or retraction of both straps. This gear-driven mechanism is what allows for bidirectional tightening and loosening of the brace around the user's arm.

The upper housing 26 is placed atop the lower housing 27 and aligned and connected via six tab-and-recess connections. Tabs 64, which protrude laterally from the lower housing, are received into corresponding recesses 65 of the upper housing. There are three tab-recess pairs on each lateral side of the housing assembly. Notably, the outermost four recesses are intentionally manufactured to be wider than their corresponding tabs, allowing the upper and lower housings to move differentially relative to each other. This feature facilitates curvature adaptation when the assembled device is flexed to accommodate arms of various diameters. The thickness of the upper and lower housing components is equal, an important structural detail that promotes maximum flexure and optimal bendability of the combined housing structure across its entire span.

The material used for both the upper and lower housings is preferably a semi-rigid injection-moldable polymer, such as polypropylene (PP), which offers a balance of resilience, toughness, and flexural compliance. PP's high flexibility index enables the housing structure to adjust conformally to the contour of the user's arm without cracking or fatiguing under repeated stress. However, any suitable material may be used.

Square driver shaft 53 passes through hole 54 of upper housing 26, which is centrally located with respect to gear ring 61 and guide ring 60 which surround it. Both gear ring 61 and guide ring 60 are integrally manufactured as part of the upper housing. Rotary knob 29, which features a scalloped profile for ergonomic grip, is slid over the protruding square drive shaft 53 by means of a square central drive hole 55. A compression spring 56 is fitted between the knob and a retainer disc 57, which in turn is secured to the end of post 49 using a screw 59 that seats in the recess that surrounds hole 58, through which it passes before threading into hole 50. In normal operation, spring 56 biases knob 29 downward such that its internal gear ring 61a, shown on the reverse side of knob 29, mates with the stationary gear ring 61 molded into upper housing 26. These interacting and mating gear rings (that include, e.g., mating or meshing teeth or projections) provide a locking mechanism that prevents accidental strap motion. The knob 29 may be biased inwardly with stationary gear ring and internal gear ring mated in a normal position to lock the positioning for the proper tension in place. To unlock, disengage or unmesh and change the tension, the user must pull the knob outwardly. This outward lifting or pulling action differs from outwardly biased locking systems that require a pushing action to disengage locking, and is used in the present invention to prevent the user from rolling over during sleep onto the knob, having the knob accidentally pushed in, and accidentally loosening the arm brace.

To adjust the brace tension, the user lifts knob 29 against the force of spring 56, disengaging gear rings 61 and 61a. When the knob is rotated in this lifted state, the square post-hole interface between shaft 53 and knob 29 transmits torque to the integrated pinion gear 52, which then drives the opposing gear racks 62 and 63 to move the straps inward (tightening) or outward (loosening). Once the desired fit is achieved, releasing the knob allows the spring to re-engage the gear rings, locking the straps securely in place.

Material selection for the internal components is made with mechanical performance in mind. The rotary knob 29, pinion gear 52, and drive shaft 53 are all subjected to significant stress during tightening and are therefore fabricated from higher-strength polymers, such as polyoxymethylene (POM), polyamide (PA), polycarbonate (PC), or thermoplastic polyester, each of which offers excellent dimensional stability, mechanical toughness, and resistance to fatigue.

The straps 20 and 21, including their integrated gear racks, require a combination of flexibility and mechanical durability, particularly in the ratcheting teeth that engage with the pinion. A preferred material for these straps is POM, which provides a superior balance of low friction, high tensile strength, and fatigue resistance, enabling long-term reliable performance under cyclical loading conditions.

FIG. 14 presents a cross-sectional and bisectional view of the rack-and-pinion tightening system, specifically illustrating the internal configuration and interface between the upper housing 26 and lower housing 27, which together form the casing that houses the mechanical components of the adjustment mechanism.

The view reveals how the two housing halves are joined together by mechanical interlock through tabs 64 and recesses 65. These tab-recess pairs serve to center, align, and securely interconnect the housings. During assembly, tabs 64, which are integrally molded into the lower housing 27, are urged into corresponding recesses 65, which are likewise integrally molded into the upper housing 26. As the housings are pressed together, tabs 64 temporarily deflect the outer walls of the upper housing outward until they pass the lip of each recess, at which point they snap into place, locking the housings into a unified structure.

Each tab 64 protrudes from the lower housing 27 by approximately 0.030 inches and has a width of around 0.250 inches. The recesses 65, which receive these tabs, are dimensioned to either closely match the width of the tab or to be wider depending on their location. Specifically, the two central recesses on each lateral side of the housing are about 0.255 to 0.265 inches wide, which provides a low-play fit that prevents unwanted lateral movement between the upper and lower housings. In contrast, the outermost four recesses, not visible in this cross-sectional view, are approximately 0.350 to 0.500 inches wide, intentionally allowing for differential relative movement of the housings. This differential movement enables the assembly to adapt to varying curvature when the brace flexes to fit arms of different diameters.

At the center of the lower housing 27 is vertical post 49, which serves as the central anchor for the pinion mechanism. Centering ring 60 and stationary gear ring 61 are integrally manufactured with upper housing 26. Stationary gear ring 61 has teeth or projections that engage with the mating gear ring of the rotary knob as described in prior figures.

One of the design elements illustrated in this figure is the relative height relationship between the upper and lower housings. Specifically, the height H2 of the upper housing 26 is shown to be equal to the height H3 of the lower housing 27. This symmetry in wall height is intentionally engineered to optimize the flexibility of the fully assembled structure. If the height of one housing were significantly greater than the other, it would result in exponentially increased resistance to flexure under bending loads. By keeping H2 and H3 equal, the design ensures uniform flexibility across the arc of the device, promoting greater conformability to varying forearm shapes and reducing stress concentrations.

This structural consideration is helpful because the housing assembly must bend and contour to the user's arm during tightening. When combined with the use of flexurally compliant polymers such as polypropylene (PP), the preferred material for the housing due to its excellent balance of strength and flexibility, the result is a brace system that delivers both mechanical integrity and comfort-focused adaptability.

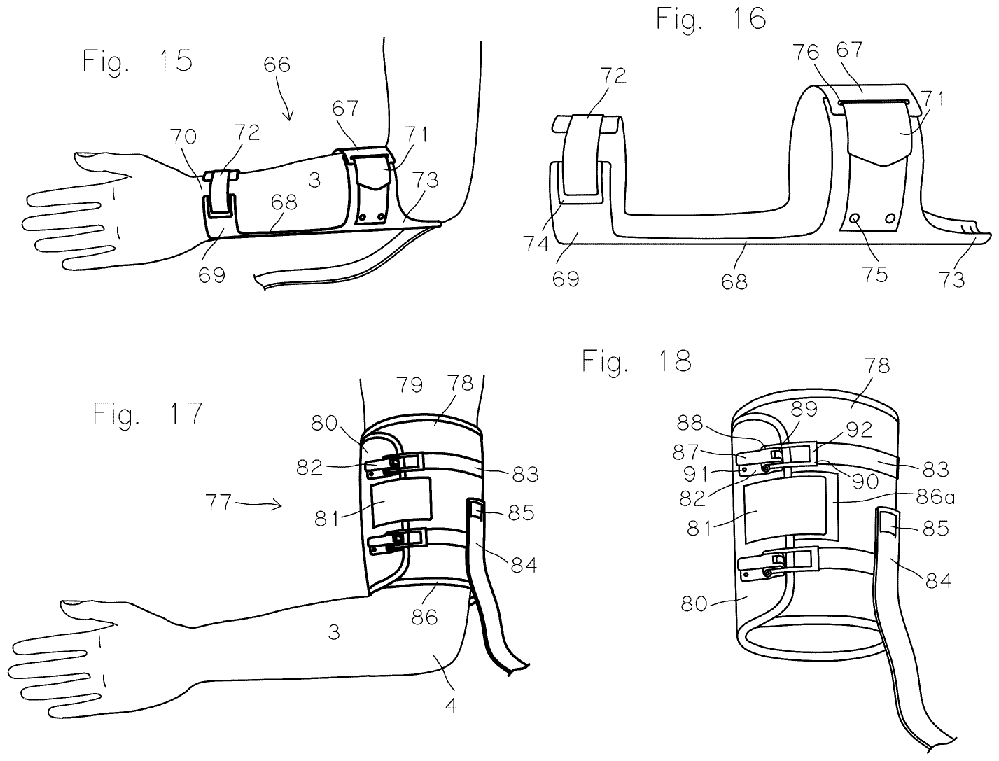

FIG. 15 and FIG. 16 illustrate another embodiment of an arm brace 66 for positioning on the user's forearm. The brace comprises an upper arm portion 67 (or first circumferential portion), a bridge or connecting extension 68, and a wrist portion or pair of laterally projecting wrist wings 69 (or second circumferential portion) that at least partially wrap around the user's wrist 70. The structure may be formed from a semi-rigid material, such as PVC, which provides structural stability while permitting enough flexibility to conform to the shape of the arm. However, this is not a limitation and any suitable material may be used.

The upper arm portion 67 at least partially encircles the forearm and may terminate in overlapping distal ends, allowing the internal diameter to be adjusted to accommodate different arm sizes. A tightening strap 71 passes through a slot 76 (shown in FIG. 16) located on the distal end of the overlapping section, then folds back and secures to itself by hook-and-loop fastener means. Because the strap is routed through the slot before fastening, it forms a looping mechanism that creates a 2-to-1 mechanical advantage, therefore a moderate pulling force on the strap results in a proportionally greater compressive force on the arm. As the strap is drawn tighter, the overlapping end of portion 67 slides further over the underlying portion, cinching the brace securely around the upper forearm. Despite being manually actuated, this system is capable of exerting significant tightening force due to the inherent leverage of the looped design. Strap 71 is affixed to the brace using securements such as rivet connections 75, which are shown in FIG. 16. Other securing means or mechanisms are within the scope of the present invention.

In contrast, the lower portion of the brace, comprising wrist wings 69, is not intended to produce circumferential compression. Rather, it is designed to remain loosely fitted to allow comfortable axial rotation of the forearm. A simple strap 72 spans the gap between the distal ends of the wrist wings and fastens to a loop patch 74 (shown in FIG. 16).

Unlike strap 71, strap 72 does not pass through any mechanical routing feature and functions as a standard hook-and-loop strap. Wings 69 serve to increase surface area contact with the forearm, increase rotational stability and help to prevent slippage of the brace past the wrist when subjected to axial traction forces. The hook and loop portions in any of the embodiments described herein are reversable.

At the proximal end of the brace, extension 73 functions as the elastic cord attachment point, and is located adjacent the elbow to align with the elbow's center of rotation, in accordance with the design principles of the invention. As further shown in FIG. 16, extension 73 incorporates two parallel cord slots through which the elastic cord is threaded. As described in prior embodiments, this double-slot configuration resists slippage through friction while allowing therapeutic tension to be maintained throughout use.

FIG. 16 presents a side view of the same arm brace 66, illustrating the relationship among its primary structural and fastening components. The upper arm portion 67 is tightened by drawing strap 71 through slot 76, then folding it back onto itself, where it adheres via hook-and-loop fastening. Strap 71 is anchored securely to a lower section of portion 67 using rivets 75, allowing the overlapping portion of the brace to be pulled taut around the user's arm.

The wrist region of the brace includes the laterally extending wings 69, which wrap around the wrist without compressing it. The wings are joined by strap 72, which fastens to a loop patch 74 affixed to the distal end of the opposite wing. This looser connection permits the forearm and wrist to rotate freely while still stabilizing the brace against unwanted movement or slippage.

Finally, extension 73, located at the end of wrist extension 68, incorporates two parallel slots to receive the elastic cord. This configuration ensures a secure and frictionally retained connection, which maintains appropriate tension while allowing freedom of forearm and hand movement in accordance with the therapeutic objectives of the invention.

FIG. 17 illustrates another embodiment of the present invention, in which an upper arm brace, generally designated as 77, is positioned on the upper arm 79 of the user. Brace wrap 78 or main body portion is configured as a wrap-around cuff with overlapping portion 80, constructed of a thick, padded cloth material, which offers a combination of comfort, flexibility, and resistance to buckling. It may additionally have a semi-rigid exterior superstructure to assist in maintaining its shape and form under tension from the elastic cord. The brace is sized and shaped to envelop the upper arm snugly while providing adequate structural integrity to support tension forces transmitted by the elastic cord.

The brace is initially configured by the user using a hook-and-loop fastener 81 or other connection mechanism, which enables the cuff to be adjusted to a size that just slips over the elbow 4 and onto the upper arm 79. Once positioned correctly on the upper arm, such that the bottom edge or rim 86 of the cuff rests securely at the top of the elbow, final tightening is achieved quickly and efficiently using flip-over-center latch mechanisms 82.

Each latch 82 is connected to an associated tightening or tensioning strap 83, which may be fabricated from either a non-stretch woven textile material, such as nylon, or an elastic band, depending on the desired degree of stretch and conformity. The latch mechanism is operated by flipping the lever arm over a “center” fulcrum point, thereby drawing the strap tight and securing the brace in place. To remove the brace, the latch is simply rotated back across the fulcrum to its release position, allowing the strap to loosen. This design enables rapid donning and removal with consistent and reproducible tightening force.

The bottom rim 86 of the cuff, which lies adjacent the elbow, includes additional integrated padding. This helps prevent localized pressure concentrations that might otherwise occur at the contact point when tension from elastic cord 84 is applied. The cord attachment point is located above rim 86 at connection 85, near the mid-height of the brace, which serves to distribute the traction force more evenly around the circumference of the upper arm and prevent localized pressure at the elbow. In this embodiment, therefore, the attachment point of elastic cord or strap 84 is not at the elbow but is above it, therefore the forearm and hand may be freely moved without restriction and with little to no torque generated at the elbow as with the other embodiments.

FIG. 18 provides a more detailed view of brace 78 and specifically the flip-over latch tightening mechanism 82. The mechanism includes a lever arm 87, a rotary latch connection joint 88, and a pivot joint 89, which serves as the fulcrum point for latch operation. The opposite end of latch 90 is connected to the tensioning strap 83 at attachment point 92, where the strap is securely wrapped or sewn around the latch.

As previously noted, straps 83 may be composed of woven textile (e.g., nylon) or elastomeric materials to provide either rigidity or elastic conformity, depending on the preferred performance characteristics. Strap 83 travels through or around brace 78 and is tightened by motion of lever 87, which pivots over fulcrum 89 to draw the strap taut.

The hook-and-loop strap 81 used to configure the slip-over fit is secured to the opposing end of the brace by attaching to loop patch 86a. Once adjusted, this strap may remain connected for reuse on future applications without needing reconfiguration. Both strap 84, which serves as the elastic cord, and the hook-and-loop strap 81 may be permanently attached to brace 78 by sewing, adhesive bonding, or other appropriate fastening methods.

FIG. 19 illustrates an embodiment of the arm brace of the present invention that is structurally similar to the embodiment previously shown in FIG. 10, with many of the same features identically numbered for clarity and continuity. This embodiment includes two knob-controlled rack and pinion tightening systems, which are mechanically joined together to operate as a single integrated unit. The purpose of this dual configuration is to expand the effective compression and contact area around the user's arm, thereby enhancing stability, pressure distribution, resistance to rotation during use and slippage down the forearm.

The brace includes strap sections 20, each of which is part of the circumference-adjustable brace body that wraps around the user's forearm. Each strap is controlled by a corresponding rotary tightening knob 29, mounted on an upper housing section 26. As with the previously described embodiments, rotation of the knobs 29 operates a rack and pinion mechanism to selectively tighten or loosen each strap section 20 (and 21, not shown), securing the brace around the arm with adjustable compression.

Also shown are key structural elements integral to the functionality of this embodiment of the brace: an ulnar support extension 22, which projects along the forearm in the direction of the wrist to provide both pressure distribution and resistance to torsional forces; and a proximal connection extension 23, which serves as the anchor point for an elastic cord (not shown), providing the dynamic traction functionality characteristic of the invention. The two independently controlled tightening units are joined via an interconnecting bridge 46, creating a unified device while preserving individual adjustability.

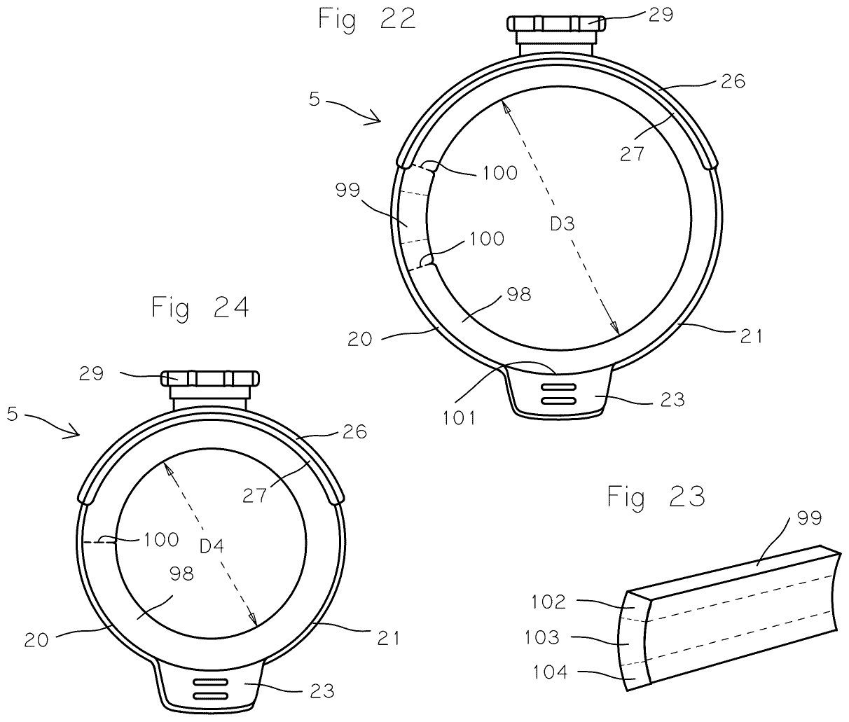

Visible within the intermediate space or gap 46a between the two rack-and-pinion systems is a compliant foam rubber padding liner 93, shown in greater detail in FIGS. 22, 23, and 24. This padding liner 93 runs continuously along the inner circumference of the brace and also covers ulnar extension 22, providing enhanced comfort and even pressure distribution. The padding is configured to extend slightly beyond the proximal and distal ends of the brace sections, ensuring complete coverage of the user's arm and minimizing localized edge pressure. When the knobs 29 are tightened, the padding compresses along with the brace structure, enabling the device to conform securely to the user's arm while maintaining cushioning.

A unique feature of this embodiment is its conically tapered configuration. The brace, as illustrated, narrows continuously from the proximal side (near the elbow) toward the distal end (toward the wrist). This design reflects the natural anatomical taper of the human forearm, which typically narrows at a 4 to 8 degree cone angle, and in the figure is represented by dotted line D-E, angled approximately 7 degrees with respect to the plane of the brace base and bridge 46.

To achieve this conical geometry, the straps 20 (and complementary straps 21) are pre-shaped during manufacturing to follow the required taper. Each rack and pinion unit retains independent control, allowing for precise adjustment along both upper and lower regions of the arm, complemented by the angled geometry. The tilted orientation of each section provides a more anatomically accurate and secure fit, improving both user comfort and the biomechanical performance of the device.

FIG. 20 and FIG. 21 depict an embodiment of the rack-and-pinion arm brace 5 in which the entire external and internal structure of the brace, excluding the adjustment mechanism, is enclosed within a stretchable fabric cover or jacket 94, preferably made from a nylon fabric-laminated neoprene material. This embodiment demonstrates how the brace can accommodate varying forearm diameters by stretching and relaxing the outer jacket in accordance with changes in brace aperture.

Referring first to FIG. 20, brace 5 is shown in a reduced diameter configuration, designated as diameter D1, which may range from approximately 3 to 5 inches. In this state, the jacket 94 is in a relaxed or non-stretched condition, closely conforming to the reduced internal circumference of the brace. The jacket surrounds all portions of the brace, including the internal circumference areas that contact the arm, providing a soft, compliant interface between the user's skin and the brace structure. The only area left exposed is the external rotary dial 29, which must remain accessible for adjustment and thus appears through cutout region 95 in the jacket.

The jacket comprises two overlapping sections on each strap side of the brace. Jacket sections 20a and 20b wrap around the underlying strap 20 (not visible in the figures), and on the opposite side of adjustment knob 29, the jacket similarly covers strap 21 (not visible in the figures), with the visible portion of the jacket section labeled 21b. The overlapping sections 20a and 20b overlap at junction 96 and are secured using three snaps 97, which are evenly spaced to maintain jacket positioning. An identical fastening arrangement exists with respect to the jacket material covering strap 21 on the opposite side of knob 29. The extension portion 22a, which corresponds to the ulnar forearm support, is also fully wrapped in neoprene. It will be appreciated that the material of the jacket slides against the material of the superstructure or main body portion when the diameter of the arm aperture is increased or decreased (i.e., when the tension is adjusted).

The selected neoprene material has highly elastic characteristics, making it well-suited to follow the expanding and contracting contours of the brace. The neoprene possesses an elongation percent greater than 400, meaning it can stretch to over four times its original length without permanent deformation or damage. Additionally, it exhibits a cell modulus at 60% strain of approximately 0.6 to 0.75 kg/cm², ensuring that it stretches easily while retaining form and offering mild resistance. While the neoprene jacket material will compress circumferentially inside the brace, its stretch characteristics are more useful as elongation prevents buckling of the material that occurs from the same or lower percentage of compression, particularly in thinner neoprene cross-sections ranging from 3 to 6 mm. If desired, perforated neoprene may be used to further increase flexibility and enhance breathability for user comfort.

In contrast, FIG. 21 shows the same brace in an expanded state, with a larger internal diameter D2 that may range from approximately 4 to 6 inches. This larger diameter is achieved by rotating the adjustment knob 29, which causes the rack and pinion system to drive the straps apart. As the circumference of the brace increases, sections 20a, 20b, (21a—not shown) and 21b stretch longitudinally and slide along the underlying straps 20 and 21 (not shown) due to radial expansion of the brace and the resulting increased lengths of the straps, with the circumferential expansion being the sole dimension of elongation. The jacket remains in continuous contact with the brace and conforms to the expanded shape due to its pre-fastened, full-wrap design and the snap attachments 97 at multiple points around the external surface.

The secure and elastic fit of jacket 94 ensures it tracks the full range of dimensional adjustments that brace 5 can make, whether the brace is tightened to a small aperture for smaller users or expanded to accommodate larger forearms. Elastic jacket 94 automatically adjusts to selected arm sizes as the user rotates adjustment knob 29.

FIG. 22, FIG. 23, and FIG. 24 depict an embodiment of arm brace 5 that features a compliant foam rubber padding liner 98, placed against the inner circumference of the brace. This inner liner offers an alternative padding solution to the neoprene fabric stretch jacket 94 described in FIGS. 20 and 21, and it is designed to maintain continuous, dynamic contact with the user's forearm through controlled compression. It will be appreciated that the inner liner may be a continuous circle or have a circular shape that is inserted into the arm aperture of the main body portion or it may be a length of material that is formed in a circle to be placed in the arm aperture (as shown in FIG. 22).

Referring to FIG. 22, foam rubber liner 98 is compressively secured against the internal surfaces of the brace or the inner surface of the main body portion, particularly along strap sections 20 and 21 and lower housing 27. The liner may be adhered to the brace at a single location 101 along the length of the liner, typically including extension 22 (not shown in this figure), but is otherwise left unattached to allow the remainder of the liner to move, compress, and expand freely. This targeted securement strategy ensures that the liner remains aligned during tightening while also maintaining full compliance with changes in brace curvature and aperture diameter.

When the brace is expanded to a larger diameter D3, typically ranging from 4 to 6 inches, the foam liner 98 is in a relatively relaxed, non-compressed state. In this configuration, the liner rests flush against the brace's interior surface but applies minimal outward force. As the brace is tightened using rotary knob 29, the internal aperture contracts, and foam liner 98 becomes compressively stressed. Because the material resists compression uniformly from all directions, it pushes outwardly against the brace walls while simultaneously conforming to the user's arm for a secure fit. Notably, compression increases radially during tightening, enhancing contact pressure in a controlled and comfortable manner.

A unique and beneficial feature of this embodiment is the use of manually removable foam insert 99, which occupies the discontinuity or gap formed at the terminal ends or first and second ends of liner 98, and stays in place due to pressure and compressive forces of the liner ends. Insert 99 increases the padded circumference to accommodate a range of larger forearms. As shown in FIG. 23, insert 99 comprises three stacked, breakaway segments or sections 102, 103, and 104 (or first section 102, second section 103 and third section 104) joined into a single modular unit. These segments may be separated via integrated perforations to tailor the width to the user's needs. The width of the insert may range from. 5 inches to 2 inches, and its length may range from 1 inch to 6 inches, depending on whether a single or double tightening system is used. Insert 99 may be entirely removed for smaller arms or when unneeded. The diameter of the main body portion is adjustable to increase and decrease the diameter of the arm aperture, when the diameter of the main body portion is a predetermined diameter (where the circumference of the inner surface of the main body portion exceeds the length of the inner liner), the gap is defined between the first and second ends of the inner liner. At this configuration, one or more inserts can be placed in the gap.

The foam rubber materials used in liner 98 and insert 99 may include fabric-laminated nitrile butadiene rubber (NBR) or similar elastomers with suitable resilience and comfort characteristics. The cross-sectional thickness of the liner may range from about ⅜ inch to ⅝ inch. Any suitable material is within the scope of the present invention.

A further combination embodiment may incorporate both foam rubber liner 98 and the external neoprene stretch jacket 94 shown in FIGS. 20 and 21, to enhance performance and comfort. In such a configuration, liner 98 is placed inside the brace, contacting its inner walls, while jacket 94 surrounds and covers liner 98 and the external surfaces of the brace. This dual-layer configuration introduces an elegant balance of opposing force vectors: the compressively stressed liner 98 resists inward movement during tightening, while the expansively stressed jacket 94 resists outward movement during loosening or enlargement. When used together, these elements create a partial cancellation of internal stresses, thereby reducing the net effort required by the user to tighten or loosen the brace via the adjustment knob. Additionally, by combining the thicknesses of both the inner foam and the outer stretch layer, the overall cushioning effect is enhanced, increasing user comfort and contact uniformity around the arm.

Referring now to FIG. 24, brace 5 is shown in a reduced diameter configuration, wherein internal diameter D4 ranges from approximately 3 to 5 inches. In this example, insert 99 is not used, and foam liner 98 alone provides the compressive interface. As diameter D4 is reduced, compression of liner 98 increases, causing the liner's cross-section to displace inwardly, thereby further adding to the reduction in aperture size.

At location 100, the terminal ends of liner 98 meet, and both frictional engagement and radial compression hold them in place without gaps, forming a continuous and sealed inner circumference. Additionally, as described in FIG. 19, liner 98 may be extended to cover distal extension 22, ensuring padding throughout the contact surface of the brace. Liner 98 may also extend to partially or fully cover the proximal extension 23, which serves as the elastic cord attachment location, further providing continuous support.

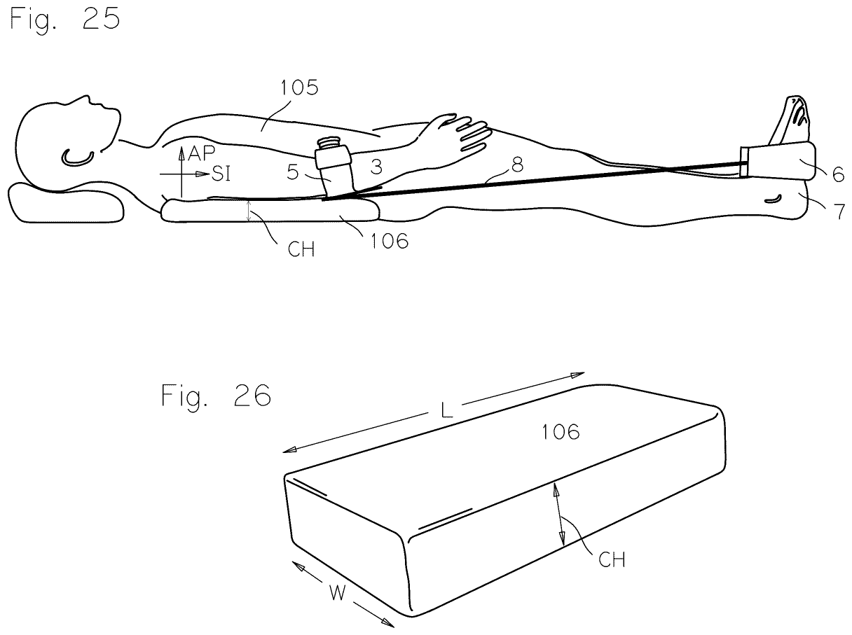

FIG. 25 is a side view of individual 105 in a supine position wearing a brace assembly including arm brace 5 on forearm 3 and foot stirrup 6 on foot 7, interconnected with elastic cord 8. The traction device or brace assembly provides tension on the homolateral arm that translates to an inferior distractive force applied to the shoulder in the superior-inferior plane SI. This inferior force, indicated by the direction of the horizontal arrow, relieves shoulder impingement compression in one of two planes of force with respect to the shoulder, the SI plane. The other plane of force is in the anterior-posterior plane AP, indicated by the vertical arrow, from the back to the front of the body. When the individual moves from a standing or sitting position to a supine position, the gravitational vector shifts by about 90 degrees, resulting in not only superior migration of the humeral head in the SI plane, but also posterior migration of tissues in the AP plane, causing severe pain. This posterior translation increases joint strain, alters the resting alignment of the glenohumeral joint, and exacerbates subacromial impingement and capsular tension. Posterior shoulder translation in the AP plane can be countered and shoulder neutrality restored by encouraging upward migration of the shoulder and upper arm with support cushion 106. The height of the cushion CH is critical in restoring neutrality and is calculated as a percent of body height of the individual. Restoring shoulder neutrality in the anterior-posterior plane is especially important for individuals suffering with thoracic outlet syndrome, where symptoms are known to worsen when the shoulder is depressed posteriorly.

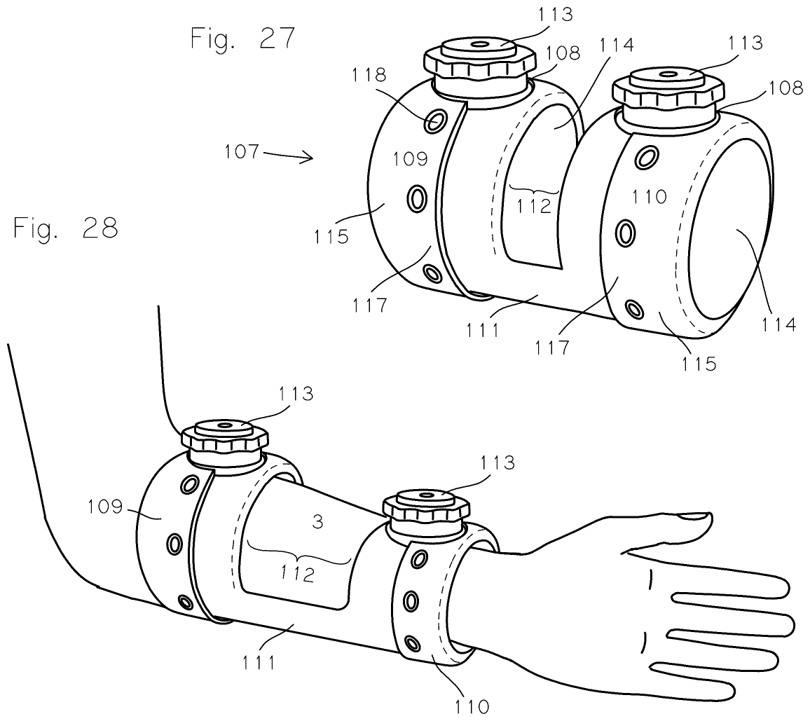

Referring now to FIG. 26, a detailed perspective view of support cushion 106 is shown. Both the height and indention load deflection (ILD) of the cushion are critical to achieving this neutral positioning and relieving pain. For individuals with an average body build, optimal support height CH is calculated as 3 to 4 percent of their body height in inches. For instance, a person standing 5 feet 10 inches (70 inches) would require a support cushion approximately 2.1 to 2.8 inches in height. The width of the cushion W may range from 3 to 10 inches (and any range therebetween) and its length L may range from 8 to 20 inches (and any range therebetween). A preferred width W is 6 inches and preferred length L is 16 inches. The cushion's ILD, a standard measure of foam firmness, should preferably be in the range of 25 to 45 to provide adequate support without discomfort. When properly placed and dimensioned, the cushion offsets the posterior pull of gravity and supports the arm in a neutral scapular and glenohumeral orientation. This, in conjunction with the inferior distraction of the shoulder applied by the elastic cord, allows the shoulder to remain comfortably decompressed in all planes while the user sleeps or rests.

Referring to FIG. 27 and FIG. 28, a brace 107 is shown in a side perspective view and includes two rack and pinion tightening systems 108 designed for use as an orthopedic brace on the arm or leg of a user. Referring to FIG. 27, brace 107, as an orthosis provides support and stabilization for joints or muscles, and may be used for conditions such as fractures, ligament injuries, deformities, muscle or joint instability, contractures, or to reduce pain. Brace 107 has features and characteristics similar to those of previous figures, particularly FIGS. 5, 10, 20 and 21, and in this embodiment comprises two brace sections 109 and 110 connected by bridge 111 and separated by space 112. Bridge 111 is generally opposite the internal rack and pinion systems and controlling knobs 113, while space 112 separates the two brace sections. The rack and pinion system utilized may be the one shown in the exploded and cross-sectional views of FIGS. 13 and 14, respectively. Brace 107 presents an orthosis with continuous and permanent apertures 114 for placement around the treated limb (which may be referred to herein as a limb aperture, an arm aperture or leg aperture). The primary benefits of brace 107 include dual independent rack and pinion tightening systems 108, quick and easy application and removal by inserting or withdrawing the limb extremity through adjustable apertures 114 to the targeted area for support, ease of tightening and adjustment via knobs 113 that control the underlying rack and pinion tightening systems 108, and elastic padding jacket 115 that frictionally slides against underlying strap members (20 and 21 in FIG. 13) and which, through adjustment by the rack and pinion system, increase or decrease in size in concert with apertures 114 to fit a variety of limb diameters. Cover or jacket 115 surrounds brace sections 109 and 110 and provides cushioned padding within the apertures 114 against the inner circumference of the brace sections where there is contact with the limb. Jacket 115 has overlapping sections 117 that affix to the underlying padding layer via six snaps 118 located on both sides of both brace sections (six snaps on the opposite sides of knobs 113 not visible).

Jacket 115 may be in a relaxed or non-stretched state when apertures 114 are adjusted to a small diameter opening. When apertures 114 are adjusted to a larger diameter opening, jacket 115 may be in a stressed and expanded state. Due to elasticity and its sliding action against the brace structure it girdles, apertures 114 of jacket 115 adjust responsively to rotation of knobs 113.

As with previous embodiments, the selected neoprene material has highly elastic characteristics, making it well-suited to follow the expanding and contracting contours of the brace. The neoprene possesses an elongation percent greater than 400 and exhibits a cell modulus at 60% strain of approximately 0.6 to 0.75 kg/cm², ensuring that it stretches easily while retaining form and offering mild resistance. While the neoprene jacket material will compress circumferentially inside the brace, its stretch characteristics are more useful as elongation prevents buckling of the material that otherwise occurs from the same or lower percentage of compression, particularly in thinner neoprene cross-sections ranging from 3 to 6 mm. If desired, perforated neoprene may be used to further increase flexibility and enhance breathability for user comfort. Bridge 111 may be fixed in length or adjustable, for example through locking adjustment means (exemplified in FIG. 31).

Referring to FIG. 28, brace 107 is positioned on forearm 3 with the bridge 111 against the ulnar side of the arm opposite adjustment knobs 113. In this case, bridge 111 has been manufactured to an appropriate length for usage on forearm 3. Resulting space 112 provides a wide, open area between brace sections 109 and 110.

To position brace 107 on the arm, apertures 114 on both sections 109 and 110 are enlarged enough to fit over the hand, and once in position, the aperture is reduced to tighten each brace section 109 and 110 around the different arm sections using knobs 113, driving the associated rack and pinion tightening systems. The human arm tapers from the elbow to the wrist area, and as such, brace sections 109 and 110 will be tightened independently and by different amounts to achieve a secure and comfortable fit of the padded brace on arm 3. As is visible in the drawing, section 110, located at the wrist area, is tightened to a greater degree and is smaller than section 109.

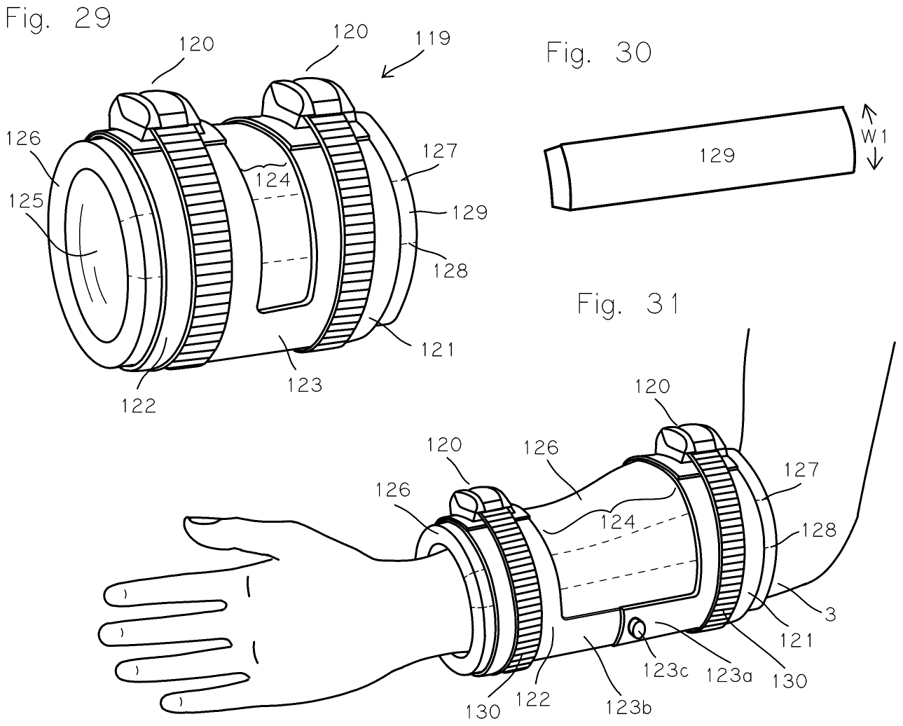

Referring to FIG. 29, FIG. 30 and FIG. 31, a brace 119 is shown in side perspective views with two ratchet-buckle tightening systems 120, designed for use as an orthopedic brace on the arm or leg of a user. Referring to FIG. 29, brace 119, as an orthosis provides support and stabilization for joints or muscles, and may be used for conditions such as fractures, ligament injuries, deformities, muscle or joint instability, contractures, or to reduce pain. The superstructure of brace 119 includes semi-rigid brace portions 121 and 122 and bridge 123 and may be produced from one of a variety of semi-rigid polymers typically used in injection or compression molding, and in this case is produced from PVC. Brace 119 has features and characteristics similar to those of previous figures, particularly FIGS. 6, 22, 23 and 24, and in this embodiment comprises two semi-rigid brace sections 121 and 122 connected by bridge 123 and separated by space 124. and located generally opposite the ratchet-buckles 120. As shown in FIG. 31, Bridge 123 may be adjustable in length through relative longitudinal movement of sections 123a and 123b. Once the desired length and fit is achieved, adjustment securing knob 123c is tightened to cinch and lock the two sections against one another. The ratchet-buckle tightening system 120 may be similar or identical to that shown and described with respect to FIG. 6. Brace 119 presents an orthosis with continuous and permanent apertures 125 for placement around the treated limb. The primary benefits of brace 119 include dual independent ratchet buckle tightening systems 120, quick and easy application and removal by inserting or withdrawing the limb extremity through adjustable apertures 125 to the targeted area for support, ease of tightening and adjustment using ratchet lever of buckles 120, and a foam rubber padding liner 126 that circumferentially fits against the inside surface of the strap members comprising sections 121 and 122 (shown similarly as 20a and 21a of FIG. 6) which, by means of manual adjustment of the ratchet-buckle systems, increases or decreases in size in concert with adjustment of apertures 125 to fit a variety of limb diameters. Liner 126 has terminal ends 127 and 128 that either abut one another or abut removable enlargement insert 129. Liner 126 is adhesively secured along an inner length of the semi-rigid superstructure that extends across bridge 123 to include portions of brace sections 121 and 122, preferably opposite ratchet buckles 120, and in the remaining areas is secured in place against the brace through compression by tightening of the brace around a limb. Liner 126 may be in a generally relaxed or non-compressed state when apertures 125 are adjusted to a large diameter opening, and in this state applies minimal outward force against the inner brace surface. When apertures 125 are adjusted to a smaller diameter opening using the ratchet system 120, liner 126 may be in an elastically compressed state, pushing outwardly against the inner brace surface while displacing inwardly to increase the cross-sectional thickness of the liner, further reducing the aperture diameter.

A unique and beneficial feature of this embodiment is the use of manually removable foam insert 129, which occupies the discontinuity formed at the liner's terminal ends 127 and 128, and stays in place due to pressure and compressive forces of the terminal ends. Insert 129 increases the padded circumference to accommodate a range of larger forearms. For smaller arms, insert 129 may be removed, in which case terminal ends 127 and 128 will abut one another. FIG. 30 shows a side perspective view of insert 129, which may range from 0.5 inches to 2 inches in width W1, while its length and thickness should equal that of the liner 126, but also may include different dimensions.

The foam rubber materials used in liner 126 and insert 129 may include fabric-laminated nitrile butadiene rubber (NBR) or similar elastomers with suitable resilience and comfort characteristics. The cross-sectional thickness of the liner may range from about ⅜ inch to ⅝ inch.

A further combination embodiment may incorporate both foam rubber liner 126 and the external neoprene stretch jacket 115 shown in FIGS. 27 and 28, to enhance performance and comfort. In such a configuration, liner 126 is adhesively affixed in place inside the brace as previously described, with the non-affixed portions conforming to its inner wall circumference, while jacket 115 surrounds and covers liner 126 and the external surfaces of the brace. This dual-layer configuration introduces an elegant balance of opposing force vectors: the compressively stressed liner 126 resists inward movement during tightening, while the expansively stressed jacket 115 resists outward movement during loosening or enlargement. When used together, these elements create a partial cancellation of internal stresses, thereby reducing the net effort required by the user to tighten or loosen the brace via the ratchet buckle system. Additionally, by combining the thicknesses of both the inner foam and the outer stretch layer, the overall cushioning effect is enhanced, increasing user comfort and contact uniformity around the arm. Another combination embodiment comprises liner 126 covered with a thinner non-rubber jacket material, such as Spandex® or other compositions consisting of Polyurethane and nylon or polyester that provide an enhanced level of elasticity and breathability.

As with previous embodiments, the selected neoprene material has highly elastic characteristics, making it well-suited to follow the expanding and contracting contours of the brace. The neoprene possesses an elongation percent greater than 400 and exhibits a cell modulus at 60% strain of approximately 0.6 to 0.75 kg/cm², ensuring that it stretches easily while retaining form and offering mild resistance. While the neoprene jacket material will compress circumferentially inside the brace, its stretch characteristics are more useful as elongation prevents buckling of the material that otherwise occurs from the same or lower percentage of compression, particularly in thinner neoprene cross-sections ranging from 3 to 6 mm. If desired, perforated neoprene may be used to further increase flexibility and enhance breathability for user comfort.

Referring to FIG. 31, brace 119 is positioned on forearm 3 with the bridge portion against the ulnar side of the arm opposite adjustment buckles 120. As previously state, bridge 123 is adjustable in length through relative longitudinal movement of sections 123a and 123b. Once the desired length and fit is achieved, adjustment securing knob 123c is tightened to cinch and lock the two sections against one another. Resulting space 124 provides a wide, open area between brace sections 121 and 122, and in this case shows liner 126 extending uninterrupted between sections 121 and 122. Alternatively, liner 126 may provide padding only within the areas defined by brace sections 121 and 122.

To position brace 119 on the arm 3, apertures 125 of both sections 121 and 122 are enlarged enough to fit over the hand. Once moved to the desired position, the apertures are individually reduced in size and tightened around corresponding arm sections using the ratchet buckles 120, incrementally ratcheting along corresponding ratchet straps 130. The human arm tapers from the elbow to the wrist area, and as such, brace sections 121 and 122 will be tightened independently and by different amounts to achieve a secure and comfortable fit of the padded brace on arm 3. As is visible in the drawing, section 122, located at the wrist area, is tightened to a greater degree and is smaller than section 121.

It will be appreciated that all embodiments are interchangeable with one another and any components disclosed with one embodiment may be usable or included in any other embodiment.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, 96, other aspects may likewise be embodied as a means-plus-function claim, or in other forms, such as being embodied in a computer-readable medium. (Any claims intended to be treated under 35 U.S.C. § 112, 9 ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A brace assembly for applying traction to a shoulder of a user in a supine position, the brace assembly comprising:
an arm brace configured to be secured to an arm homolateral to the shoulder of the user, wherein the arm includes an elbow joint, a forearm and an upper arm,
a lower body anchor configured to be secured to a leg of the user, and
an elastic cord having a first end secured to the arm brace at a near connection point and a second end secured to the lower body anchor at a far connection point, wherein the elastic cord is configured to apply inferior traction to the upper arm, whereby distractive force is transmitted to a shoulder joint of the shoulder,
wherein the arm brace is configured to position the near connection point adjacent to the elbow joint, thereby allowing forearm abduction and flexion of the elbow joint while preventing abduction of the upper arm and minimizing torque at the elbow joint during forearm abduction.

2. The brace assembly of claim 1 wherein the arm brace includes a main body portion that defines an arm aperture configured to receive a user's arm, wherein a connection extension extends outwardly from the main body portion, wherein the main body portion includes a tightening system, and wherein the near connection point is located on the connection extension.

3. The brace assembly of claim 2 wherein a support extension extends outwardly from the main body portion of the arm brace in a direction opposite of the connection extension.

4. The brace assembly of claim 2 wherein the connection extension includes first and second slots defined therein, and wherein the elastic cord extends through the first and second slots to connect the first end of the elastic cord to the connection extension.

5. The brace assembly of claim 2 wherein the main body portion of the arm brace includes an upper portion and a base portion, and wherein the upper portion is pivotable with respect to the base portion.

6. The brace assembly of claim 2 further comprising a jacket that at least partially surrounds the main body portion, wherein the jacket comprises a material that is under increasing tensile stress when a diameter of the arm aperture increases and transitions toward a neutral unstressed state when the diameter of the arm aperture decreases.

7. The brace assembly of claim 2 wherein the main body portion of the arm brace is a first circumferential member with a first arm aperture, wherein the arm brace includes a second circumferential member that is spaced from the first circumferential member and that defines a second arm aperture, wherein a bridge extends between the first and second circumferential members, wherein the first circumferential portion includes a first tightening system, and wherein the second circumferential portion includes a second tightening system.

8. The brace assembly of claim 2 wherein the diameter of the main body portion is adjustable to increase and decrease the diameter of the arm aperture, wherein the arm brace further comprises an elastic inner liner positioned in the arm aperture and adjacent an inner surface of the main body portion, wherein the inner liner is under increasing compressive stress when a diameter of the arm aperture decreases and transitions toward a neutral unstressed state when the diameter of the arm aperture increases.

9. The brace assembly of claim 8 wherein the inner liner includes first and second opposite ends, wherein a gap is defined between the first and second ends of the inner liner, and wherein an insert is positioned in the gap.

10. The brace assembly of claim 1 wherein the lower body anchor is a foot stirrup, wherein the foot stirrup includes a strap member that defines a foot opening and includes a plantar portion.

11. The brace assembly of claim 10 wherein the elastic cord is at least thirty inches in length in an unstretched state.

12. The brace assembly of claim 1 wherein the elastic cord includes at least a first cord tension identifying marker that is configured to be registered with a brace tension identifying marker.

13. The brace assembly of claim 1 wherein the elastic cord has a traction tension value measured in pounds-force, wherein a torque value measured in inch-pounds is equal to or less than two times the traction tension value.

14. A brace assembly for applying traction to a shoulder of a user in a supine position, the brace assembly comprising:
an arm brace configured to be secured to an arm of the user homolateral to the shoulder, wherein the arm includes an elbow joint, a forearm and an upper arm,
a lower body anchor configured to be secured to a leg of the user, and
an elastic cord having a first end secured to the arm brace at a near connection point and a second end secured to the lower body anchor at a far connection point, wherein the elastic cord is configured to apply inferior traction to the upper arm, whereby distractive force is transmitted to a shoulder joint of the shoulder,
wherein the arm brace is configured to be positioned on the upper arm with the near connection point at or above the elbow joint, thereby allowing forearm abduction and flexion of the elbow joint while preventing abduction of the upper arm.

15. A method of applying traction to a shoulder of a user in a supine position, the method comprising the steps of:
(a) securing an arm brace to an arm homolateral to the shoulder of the user,
(b) securing a lower body anchor to a leg of the user, wherein an elastic cord that includes first and second ends extends between the arm brace and the lower body anchor, wherein the first end of the elastic cord connects to the arm brace at a near connection point that is located adjacent to an elbow joint of the user, and
(c) tensioning the elastic cord to apply inferior traction to an upper arm of the arm, whereby distractive force is transmitted to the shoulder joint.

16. The method of claim 15 wherein the elastic cord has a tension value, and wherein the tension value is between about 3 lbf and about 9 lbf.

17. The method of claim 15 wherein the lower body anchor is a foot stirrup, wherein the foot stirrup includes a strap member that defines a foot opening and includes a plantar portion, and wherein step (b) includes inserting a foot of the leg through the foot opening and positioning the plantar portion below a bottom of the foot, wherein the second end of the elastic cord connects to the strap member at a point above a dorsal surface of the foot.

18. The method of claim 15 wherein the lower body anchor includes a main body portion that surrounds the leg, wherein the second end of the elastic cord is connected to the main body portion.

19. The method of claim 15 wherein the arm brace includes a main body portion that extends around the arm, wherein the arm includes an inner side and an outer side, wherein a connection extension extends outwardly from the main body portion along the outer side of the arm and toward the elbow joint, and wherein the near connection point is located on the connection extension.

20. The method of claim 19 wherein the connection extension includes first and second slots defined therein, wherein the elastic cord includes at least a first tension identifying marker thereon, wherein the method includes extending the elastic cord through the first slot, registering the first tension identifying marker with the first slot, extending the elastic cord through the second slot.

21. The method of claim 19 wherein a support extension extends outwardly from the main body portion along the outer side of the arm and toward the wrist.

22. The method of claim 15 wherein the user has a shoulder condition or has had a shoulder surgery procedure, and wherein the shoulder condition or shoulder surgery procedure is one of a rotator cuff allograft augmentation, a shoulder osteoarthritis procedure, a shoulder replacement, acromioclavicular joint reconstruction, impingement syndrome, adhesive capsulitis or thoracic outlet syndrome.

23. The method of claim 22 wherein the shoulder surgery procedure is rotator cuff allograft augmentation that includes a decellularized dermal allograft that has been positioned between a humeral head and an acromion of the shoulder, wherein the application of inferior traction to the upper arm provides rostral distraction of the humeral head, thereby providing decompression of the decellularized dermal allograft from the acromion.

24. The method of claim 22 wherein the shoulder condition is thoracic outlet syndrome including compression of a brachial plexus in a thoracic outlet, wherein the application of inferior traction to the upper arm provides distraction of the thoracic outlet and decompression of the brachial plexus.

25. The method of claim 15 further comprising abducting the forearm and flexing the elbow joint, whereby torque at the elbow joint is minimized and the arm brace prevents abduction of the upper arm.

26. The method of claim 17 further comprising the step of reducing tension on the elastic cord by flexing the leg.

27. The method of claim 15 wherein the elastic cord includes at least a first cord tension identifying marker thereon, wherein the method includes registering the first cord tension identifying marker with a brace tension identifying marker when connecting the first end of the elastic cord at the near connection point prior to step (a).

28. The method of claim 15 further comprising positioning a cushion to support the shoulder and upper arm to mitigate posterior migration of the shoulder and to provide a neutral support for the shoulder, and wherein the thickness of the cushion is between 3 and 4 percent of the user's height.

29. An orthopedic brace for an arm or leg of a user that provides support for joints or muscles, the orthopedic brace comprising:
a semi-rigid structure, padding, and a tightening system,
wherein the brace includes a main body portion that defines an aperture configured to receive the user's arm or leg,
wherein the brace is used to treat conditions selected from the group consisting of fractures, ligament injuries, deformities, muscle or joint instability, contractures, or to reduce pain,
wherein the padding comprises
a) a compliant jacket that at least partially surrounds the main body portion and which is configured to be under increasing tensile stress when a diameter of the aperture increases and is configured to transition toward a neutral unstressed state when the diameter of the aperture decreases, or
b) a compliant liner adjacent an inner surface of the main body portion that is under increasing compressive stress when the diameter of the aperture decreases and transitions toward a neutral unstressed state when the diameter of the aperture increases, or
c) both the compliant jacket of a) and the compliant liner of b),
wherein the tightening system is selected from the group consisting of hook-and-loop, rack and pinion, ratchet buckle, cam lock, lace-and-pulley, over-center lever or toggle clamp, ratchet strap with lever release, cord lock and cinch system, dial tightening system and tensioning band with slide adjuster.
